(19) United States
(12) Patent Application Publication
(10) Pub. No.: US 2022/0193224 A1
(43) Pub. Date: Jun. 23, 2022

(54) RSV-BASED VIRUS-LIKE PARTICLES AND METHODS OF PRODUCTION AND USE THEREOF

(71) Applicant: The Board of Regents for the Oklahoma Agricultural and Mechanical Colleges, Stillwater, OK (US)

(72) Inventor: Antonius Oomens, Stillwater, OK (US)

(21) Appl. No.: 17/557,887

(22) Filed: Dec. 21, 2021

Related U.S. Application Data
(60) Provisional application No. 63/129,723, filed on Dec. 23, 2020.

(51) Int. Cl. A61K 39/155; C12N 7/00; C07K 14/005; A61P 37/04; A61P 31/14

(52) U.S. Cl. CPC A61K 39/155; C12N 7/00; A61K 2039/5254; A61P 37/04; A61P 31/14; C07K 14/005

(57) ABSTRACT

Respiratory syncytial virus (RSV)-based virus-like particles are disclosed. Also disclosed are polynucleotides encoding the virus-like particles (VLPs) as well as immunogenic compositions, pharmaceutical compositions, vaccines, and kits containing the virus-like particles. In addition, methods of producing and using each of the above compositions are also disclosed. Methods of use include single or combination administration of the RSV-VLPs, as well as use of the RSV-VLPs alone or in combination with other types of vaccines.

### CROSS REFERENCE TO RELATED APPLICATIONS/INCORPORATION BY REFERENCE STATEMENT

This application claims benefit under 35 USC § 119(e) of provisional application U.S. Ser. No. 63/129,723, filed Dec. 23, 2020. The entire contents of the above-referenced patent application(s) are hereby expressly incorporated herein by reference.

### STATEMENT REGARDING FEDERALLY SPONSORED RESEARCH OR DEVELOPMENT

This invention was made with Government support under Grant No. 1R21A1149022-01 awarded by the National Institutes of Health. The Government has certain rights in the invention

### BACKGROUND

Respiratory Syncytial Virus (RSV) is the single largest viral cause of pediatric bronchiolitis and pneumonia, with a high worldwide morbidity and mortality among not only children but also the elderly and immunocompromised populations. In spite of many years of clinical trials and scientific progress, a safe and effective vaccine against RSV has still not been found. In the 1960s, a formalin-inactivated RSV vaccine (FI-RSV) induced an imbalance in the immune response which led to enhanced pathology after exposure to wild type RSV (known as vaccine-enhanced disease (VED)). Since then, achieving an efficacious vaccine that is also safe has proven enormously challenging. Absent of an approved vaccine, both live and non-live vaccine approaches have been pursued, with each approach presenting unique qualities and hurdles.

Ever since this encounter with VED, various different vaccine platforms, including live and non-live vaccine approaches, have been evaluated; however, it has been enormously challenging to impart both sufficient safety and efficacy in a single vaccine.

One non-live vaccine approach includes the use of virus-like-particles (VLPs), which are particles that include certain structural proteins from a virus (such as outer coat proteins that allow the VLPs to mimic the organization and conformation of authentic native viruses) but do not contain any genetic material from the virus (and thus cannot cause an infection). VLPs are gaining traction, due to successes with commercial VLP vaccines and recent reports showing protective anti-RSV immunity without VED and with improved memory in animal models using VLPs without adjuvants. In part because little is known about RSV particle assembly, all VLP approaches currently in preclinical trials are based on heterologous VLP systems, with most expressing the RSV fusion (F) protein, one of two major RSV glycoproteins.

There is a need in the art for new and improved RSV vaccines that overcome the disadvantages and defects of the prior art. It is to such new and improved vaccines, as well as methods of production and use thereof, that the present disclosure is directed.

### BRIEF DESCRIPTION OF THE DRAWINGS

FIG. 1 illustrates the structure of the RSV genome along with the structures of various RSV-based virus-like particles (RSV-VLPs) constructed in accordance with the present disclosure.

FIG. 2 illustrates an analysis of RSV structural proteins/variants/fragments present in RSV-based VLPs generated in accordance with the present disclosure.

FIG. 3 graphically depicts a native cell ELISA of unfixed Fstem/CCR-G, for determining the optimal GCR C-terminus. The following constructs were compared: 137-216 (X23), 137-211 (X22), 137-206 (X8), 137-203 (X54), and 137-200 (X55). The negative control was T790 (Fstem without GCR), and the positive control was T972 (full G protein).

FIG. 4 graphically depicts a native cell ELISA of unfixed Fstem/CCR-G for determining the optimal GCR N-terminus. The following constructs were compared: 137-206 (X8), 146-206 (X7), 156-206 (T943), and 163-206 (T966). The negative control was T790 (Fstem without GCR), and the positive control was T972 (full G protein).

FIG. 5 graphically depicts a native cell ELISA of unfixed Fstem/CCR-G for determining the optimal GCR. The following constructs were compared: 156-203 (X326), 137-211 (X22), and 137-211-AA573/574 (X101). The negative control was T790 (Fstem without GCR), and the positive control was T972 (full G protein).

FIG. 6 illustrates a western analysis of RSV VLP levels carrying Fstem/GCRs with various GCR lengths, to determine most optimal Fstem/CCRG construct. The analysis utilized P, M, and G Ab 131-2G. CCRG constructs analyzed included 156-203, 156-206, 156-211, 137-203, 137-206, 137-211, and 137-211 with AA573/574, along with Fstem without GCR.

FIG. 7 contains scanning electromicrographs of VLP-GCR with G amino acids 137-211 for analysis of Fstem/CCRG particle at cell surface. Scans are of the same area. SE represents the typical scanning EM image. BSE shows gold particles (white dots) bound to GCR. The sample was incubated with anti-GCR Ab L9 and subsequently with a goat-anti-mouse Ab conjugated to 10 nm gold particles.

FIG. 8 graphically depicts that a combination vaccine of preF and GCR VLPs (VLP-preF+VLP-GCR), as well as a VLP containing both preF and GCR in one particle (VLP-preF/GCR) induce significant levels of antiviral antibodies. Different VLP compositions were used to vaccinate mice in a prime-boost regimen. Three weeks post boost, blood samples were taken, and serum antibodies against preF, whole G, and GCR were measured.

FIG. 9 graphically depicts that a prime-boost vaccine regimen of M-null and VLPs induce significant levels of anti-preF antibodies. M-null and two different VLP compositions were used to vaccinate mice in a prime-boost regimen. Three weeks post boost, blood samples were taken, and serum antibodies against preF were measured.

FIG. 10 graphically depicts that a prime-boost vaccine regimen of M-null and VLPs induce significant levels of anti-whole G antibodies. M-null and two different VLP compositions were used to vaccinate mice in a prime-boost regimen. Three weeks post boost, blood samples were taken, and serum antibodies against whole G were measured.

FIG. 11 graphically depicts that a prime-boost vaccine regimen of M-null and VLPs induce significant levels of anti-G-CCR antibodies but not anti-non-G-CCR antibodies. M-null and two different VLP compositions were used to vaccinate mice in a prime-boost regimen. Three weeks post boost, blood samples were taken, and serum antibodies against G-CCR and non-G-CCR were measured.

FIG. 12 graphically depicts an in vitro neutralization analysis of a prime-boost vaccine regimen of M-null and VLPs.

FIG. 13 graphically depicts a prime-boost vaccine regiment of M-null and VLP-preF/GCR. A strong increase in anti-GCR antibodies and a decrease in anti-non-GCR antibodies were observed. In addition, a significant increase in the ratio of preF:postF antibodies and increased the ratio of GCR:non-GCR antibodies were observed.

### DETAILED DESCRIPTION

Before explaining at least one embodiment of the inventive concept(s) in detail by way of exemplary language and results, it is to be understood that the inventive concept(s) is not limited in its application to the details of construction and the arrangement of the components set forth in the following description. The inventive concept(s) is capable of other embodiments or of being practiced or carried out in various ways. As such, the language used herein is intended to be given the broadest possible scope and meaning; and the embodiments are meant to be exemplary—not exhaustive. Also, it is to be understood that the phraseology and terminology employed herein is for the purpose of description and should not be regarded as limiting.

Unless otherwise defined herein, scientific and technical terms used in connection with the presently disclosed inventive concept(s) shall have the meanings that are commonly understood by those of ordinary skill in the art. Further, unless otherwise required by context, singular terms shall include pluralities and plural terms shall include the singular. The foregoing techniques and procedures are generally performed according to conventional methods well known in the art and as described in various general and more specific references that are cited and discussed throughout the present specification. The nomenclatures utilized in connection with, and the laboratory procedures and techniques of, analytical chemistry, synthetic organic chemistry, and medicinal and pharmaceutical chemistry described herein are those well-known and commonly used in the art. Standard techniques are used for chemical syntheses and chemical analyses.

All patents, published patent applications, and non-patent publications mentioned in the specification are indicative of the level of skill of those skilled in the art to which this presently disclosed inventive concept(s) pertains. All patents, published patent applications, and non-patent publications referenced in any portion of this application are herein expressly incorporated by reference in their entirety to the same extent as if each individual patent or publication was specifically and individually indicated to be incorporated by reference.

All of the compositions and/or methods disclosed herein can be made and executed without undue experimentation in light of the present disclosure. While the compositions and methods of the inventive concept(s) have been described in terms of particular embodiments, it will be apparent to those of skill in the art that variations may be applied to the compositions and/or methods and in the steps or in the sequence of steps of the methods described herein without departing from the concept, spirit, and scope of the inventive concept(s). All such similar substitutions and modifications apparent to those skilled in the art are deemed to be within the spirit, scope, and concept of the inventive concept(s) as defined by the appended claims.

As utilized in accordance with the present disclosure, the following terms, unless otherwise indicated, shall be understood to have the following meanings:

The use of the term “a” or “an” when used in conjunction with the term “comprising” in the claims and/or the specification may mean “one,” but it is also consistent with the meaning of “one or more,” “at least one,” and “one or more than one.” As such, the terms “a,” “an,” and “the” include plural referents unless the context clearly indicates otherwise. Thus, for example, reference to “a compound” may refer to one or more compounds, two or more compounds, three or more compounds, four or more compounds, or greater numbers of compounds. The term “plurality” refers to “two or more.”

The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z. The use of ordinal number terminology (i.e., “first,” “second,” “third,” “fourth,” etc.) is solely for the purpose of differentiating between two or more items and is not meant to imply any sequence or order or importance to one item over another or any order of addition, for example.

The use of the term “or” in the claims is used to mean an inclusive “and/or” unless explicitly indicated to refer to alternatives only or unless the alternatives are mutually exclusive. For example, a condition “A or B” is satisfied by any of the following: A is true (or present) and B is false (or not present), A is false (or not present) and B is true (or present), and both A and B are true (or present).

As used herein, any reference to “one embodiment,” “an embodiment,” “some embodiments,” “one example,” “for example,” or “an example” means that a particular element, feature, structure, or characteristic described in connection with the embodiment is included in at least one embodiment. The appearance of the phrase “in some embodiments” or “one example” in various places in the specification is not necessarily all referring to the same embodiment, for example. Further, all references to one or more embodiments or examples are to be construed as non-limiting to the claims.

Throughout this application, the term “about” is used to indicate that a value includes the inherent variation of error for a composition/apparatus/device, the method being employed to determine the value, or the variation that exists among the study subjects. For example, but not by way of limitation, when the term “about” is utilized, the designated value may vary by plus or minus twenty percent, or fifteen percent, or twelve percent, or eleven percent, or ten percent, or nine percent, or eight percent, or seven percent, or six percent, or five percent, or four percent, or three percent, or two percent, or one percent from the specified value, as such variations are appropriate to perform the disclosed methods and as understood by persons having ordinary skill in the art.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

As used herein, the term “substantially” means that the subsequently described event or circumstance completely occurs or that the subsequently described event or circumstance occurs to a great extent or degree. For example, when associated with a particular event or circumstance, the term “substantially” means that the subsequently described event or circumstance occurs at least 80% of the time, or at least 85% of the time, or at least 90% of the time, or at least 95% of the time. For example, the term “substantially adjacent” may mean that two items are 100% adjacent to one another, or that the two items are within close proximity to one another but not 100% adjacent to one another, or that a portion of one of the two items is not 100% adjacent to the other item but is within close proximity to the other item.

The term “polypeptide” as used herein will be understood to refer to a polymer of amino acids. The polymer may include d-, I-, or artificial variants of amino acids. In addition, the term “polypeptide” will be understood to include peptides, proteins, and glycoproteins.

The term “polynucleotide” as used herein will be understood to refer to a polymer of two or more nucleotides. Nucleotides, as used herein, will be understood to include deoxyribose nucleotides and/or ribose nucleotides, as well as artificial variants thereof. The term polynucleotide also includes single-stranded and double-stranded molecules.

The terms “analog” or “variant” as used herein will be understood to refer to a variation of the normal or standard form or the wild-type form of molecules. For polypeptides or polynucleotides, an analog may be a variant (polymorphism), a mutant, and/or a naturally or artificially chemically modified version of the wild-type polynucleotide (including combinations of the above). Such analogs may have higher, full, intermediate, or lower activity than the normal form of the molecule, or no activity at all. Alternatively, and/or in addition thereto, for a chemical, an analog may be any structure that has the desired functionalities (including alterations or substitutions in the core moiety), even if comprised of different atoms or isomeric arrangements.

In particular (but non-limiting) embodiments, the term “variant” as used herein may refer to a nucleotide or amino acid sequence that differs from the native nucleotide or amino acid sequence by the addition, deletion, and/or substitution of one or more residues such that the variant differs from the native sequence by less than about 25%, or less than about 20%, or less than about 19%, or less than about 18%, or less than about 17% or less than about 16%, or less than about 15%, or less than about 14%, or less than about 13%, or less than about 12%, or less than about 11%, or less than about 10%, or less than about 9%, or less than about 8%, or less than about 7%, or less than about 6%, or less than about 5%, or less than about 4%, or less than about 3%, or less than about 2%, or less than about 1%. In other particular (but non-limiting) embodiments, the term “variant” as used herein may include a nucleotide or amino acid sequence that differs from the native sequence (via additions, deletions, and/or substitutions) of less than about 20 residues, less than about 19 residues, less than about 18 residues, less than about 17 residues, less than about 16 residues, less than about 15 residues, less than about 14 residues, less than about 13 residues, less than about 12 residues, less than about 11 residues, less than about 10 residues, less than about 9 residues, less than about 8 residues, less than about 7 residues, less than about 6 residues, less than about 5 residues, less than about 4 residues, less than about 3 residues, less than about 2 residues, or about 1 residue, so long as the variant is at least about 75% identical to the native sequence.

As used herein, the phrases “associated with” and “coupled to” include both direct association/binding of two moieties to one another as well as indirect association/binding of two moieties to one another. Non-limiting examples of associations/couplings include covalent binding of one moiety to another moiety either by a direct bond or through a spacer group, non-covalent binding of one moiety to another moiety either directly or by means of specific binding pair members bound to the moieties, incorporation of one moiety into another moiety such as by dissolving one moiety in another moiety or by synthesis, and coating one moiety on another moiety, for example.

As used herein, “substantially pure” means an object species is the predominant species present (i.e., on a molar basis it is more abundant than any other individual species in the composition), and preferably a substantially purified fraction is a composition wherein the object species comprises at least about 50 percent (on a molar basis) of all macromolecular species present. Generally, a substantially pure composition will comprise more than about 80 percent of all macromolecular species present in the composition, more preferably more than about 85%, 90%, 95%, and 99%. Most preferably, the object species is purified to essential homogeneity (contaminant species cannot be detected in the composition by conventional detection methods) wherein the composition consists essentially of a single macromolecular species.

The term “pharmaceutically acceptable” refers to compounds and compositions which are suitable for administration to humans and/or animals without undue adverse side effects such as (but not limited to) toxicity, irritation, and/or allergic response commensurate with a reasonable benefit/risk ratio.

The term “patient” as used herein includes human and veterinary subjects. “Mammal” for purposes of treatment refers to any animal classified as a mammal, including (but not limited to) humans, domestic and farm animals, nonhuman primates, and any other animal that has mammary tissue.

The term “child” is meant to refer to a human individual who would be recognized by one of skill in the art as an infant, toddler, etc., or an individual less than about 18 years of age, usually less than about 16 years of age, usually less than about 14 years of age, or even less (e.g., from newborn to about 2, about 3, about 4, about 5, about 6, about 7, about 8, about 9, about 10, about 11, or about 12 years of age). The term “elderly” generally refers to a human individual whose age is greater than about 50 years of age, usually greater than about 55 years of age, frequently greater than about 60 years of age or more (e.g., about 65 years of age and upwards).

The term “treatment” refers to both therapeutic treatment and prophylactic or preventative measures. Those in need of treatment include, but are not limited to, individuals already having a particular condition/disease/infection as well as individuals who are at risk of acquiring a particular condition/disease/infection (e.g., those needing prophylactic/preventative measures). The term “treating” refers to administering an agent to a patient for therapeutic and/or prophylactic/preventative purposes.

A “therapeutic composition” or “pharmaceutical composition” refers to an agent that may be administered in vivo to bring about a therapeutic and/or prophylactic/preventative effect.

Administering a therapeutically effective amount or prophylactically effective amount is intended to provide a therapeutic benefit in the treatment, prevention, and/or management of a disease, condition, and/or infection. The specific amount that is therapeutically effective can be readily determined by the ordinary medical practitioner, and can vary depending on factors known in the art, such as (but not limited to) the type of condition/disease/infection, the patient's history and age, the stage of the condition/disease/infection, and the co-administration of other agents.

The term “effective amount” refers to an amount of a biologically active molecule or conjugate or derivative thereof sufficient to exhibit a detectable therapeutic effect without undue adverse side effects (such as (but not limited to) toxicity, irritation, and allergic response) commensurate with a reasonable benefit/risk ratio when used in the manner of the inventive concept(s). The therapeutic effect may include, for example but not by way of limitation, preventing, inhibiting, or reducing the occurrence of infection by or growth of microbes and/or opportunistic infections. The effective amount for a subject will depend upon the type of subject, the subject's size and health, the nature and severity of the condition/disease/infection to be treated, the method of administration, the duration of treatment, the nature of concurrent therapy (if any), the specific formulations employed, and the like. Thus, it is not possible to specify an exact effective amount in advance. However, the effective amount for a given situation can be determined by one of ordinary skill in the art using routine experimentation based on the information provided herein.

As used herein, the term “concurrent therapy” is used interchangeably with the terms “combination therapy” and “adjunct therapy,” and will be understood to mean that the patient in need of treatment is treated or given another drug for the disease/infection in conjunction with the pharmaceutical compositions of the present disclosure. This concurrent therapy can be sequential therapy, where the patient is treated first with one pharmaceutical composition and then the other pharmaceutical composition, or the two pharmaceutical compositions are given simultaneously.

The terms “administration” and “administering,” as used herein, will be understood to include all routes of administration known in the art, including but not limited to, oral, topical, transdermal, parenteral, subcutaneous, intranasal, mucosal, intramuscular, intraperitoneal, intravitreal, and intravenous routes, and including both local and systemic applications. In addition, the compositions of the present disclosure (and/or the methods of administration of same) may be designed to provide delayed, controlled, or sustained release using formulation techniques which are well known in the art.

Turning now to the inventive concept(s), certain non-limiting embodiments of the present disclosure are directed to a respiratory syncytial virus-based virus-like particle (RSV-VLP) that comprises an RSV phosphoprotein (P) or variant or fragment thereof, an RSV matrix (M) protein or variant or fragment thereof, and an RSV attachment glycoprotein (G) or variant or fragment thereof. The RSV G protein may be a full-length protein or variant thereof; alternatively, the G protein may be a fragment of the RSV G protein (or a variant thereof). When the G protein/variant/fragment is a fragment of G protein, the fragment comprises at least a portion of a central conserved region (CCR) of the G protein. The CCR of G protein is a 14 amino acid sequence that includes amino acids 173-186 of the G protein sequence (CSICSNNPTCWAIC, SEQ ID NO:1); the CCR contains the important CX₃C receptor binding domain.

In certain particular (but non-limiting) embodiments, the G protein/variant/fragment of the RSV-VLP is a fragment (or a variant of a fragment) of G protein, and the fragment (or variant thereof) comprises at least the CX₃C receptor binding domain or a variant thereof. For example, but not by way of limitation, an additional amino acid may be added between the two cysteines to provide a CX₄C domain, and the G protein/variant/fragment of the RSV-VLP may comprise this CX₄C domain.

In certain particular (but non-limiting) embodiments, the G protein/variant/fragment of the RSV-VLP comprises the CCR of SEQ ID NO:1 (or a variant thereof). In a particular (but non-limiting) embodiment, the G protein/variant/fragment may further comprise SEQ ID NO:2 (or a variant thereof) in addition to SEQ ID NO:1 (or a variant thereof). SEQ ID NO:2 is a heparin-binding domain (SEQ ID NO:2 is ICKRIPNKKPGKKT, amino acids 184-198 of the G protein sequence; Feldman et al. (J Virol (1999) 73(8):6610-6617)).

In certain non-limiting embodiments, the G protein or variant or fragment thereof comprises SEQ ID NO:3 or a variant thereof. SEQ ID NO:3 contains amino acids 137-211 of the G protein sequence and has the sequence of TTTQTQPSKPTTKQRQNKPPSKPNNDFHFEVFNFVPCSICSNNPTCWAICKRIPNKKPGKKTTTKPTKKPTLKTT.

In certain non-limiting embodiments, when the RSV-based VLP includes a G protein fragment (or variant thereof), the G protein fragment is fused to a polypeptide comprising a stem/stalk region of a transmembrane protein, such as (but not limited to) a polypeptide comprising a signal peptide and membrane anchor. For example (but not by way of limitation), the G protein or variant or fragment thereof is fused to a stem/stalk region of an RSV fusion (F) protein variant or fragment thereof. In another non-limiting example, the G protein or variant or fragment thereof is fused to a stem/stalk region of human NGFR (nerve growth factor receptor, a human surface protein) or any other membrane protein that can preset the G fragment on the cell surface.

The RSV-based virus-like particles may include other RSV structural proteins or variants or fragments thereof, as are well known in the art. For example, but not by way limitation, the RSV-based virus-like particles may include an RSV nucleoprotein (N) or variant or fragment thereof. Alternatively (and/or in addition thereto), the RSV-based VLPs may include an RSV fusion (F) protein variant or fragment thereof.

It has recently been recognized that the viral fusion (F) protein is unstable and readily shifts to the post-fusion conformation during purification or vaccine preparation. As a result, a large proportion of vaccine-induced antibodies (Abs) target the post-fusion form, which is functionally obsolete. To avoid induction of anti-post-fusion F Abs, the pre-fusion form (referred to as F^{PRE} or preF) has been genetically stabilized, thereby greatly increasing neutralizing capacity of anti-F Abs when given as a protein vaccine (see, for example, US Patent Application Publication Nos. US 2015/0030622 (published Jan. 29, 2015 to Marshall et al.); US 2016/0031972 (published Feb. 4, 2016 to Zheng et al.); and US 2016/0046675 (published Feb. 18, 2016 to Kwong et al.); the entire contents of each of which are hereby expressly incorporated herein by reference).

Therefore, when the RSV-based virus-like particles include an RSV F protein variant/fragment, the RSV F protein variant or fragment thereof may contain at least one mutation that stabilizes the F protein in pre-fusion form. That is, respiratory syncytial virus (RSV) F protein variant or fragment thereof includes at least one amino acid substitution when compared to a native RSV F protein, wherein the at least one amino acid substitution stabilizes the RSV F protein variant or fragment thereof in a pre-fusion conformation. Any RSV F protein variant or fragment thereof known in the art or otherwise contemplated herein may be utilized in accordance with the present disclosure, so long as the RSV F protein variant or fragment thereof includes at least one amino acid substitution compared to a native RSV F protein that stabilizes the RSV F protein variant or fragment thereof in a pre-fusion conformation. Any amino acid substitution(s) capable of stabilizing the RSV F protein variant/fragment in the pre-fusion confirmation may be utilized in accordance with the present disclosure.

Non-limiting examples of RSV F protein variants or fragments thereof (that contain one or more amino acid substitution(s) capable of stabilizing the RSV F protein variant/fragment in the pre-fusion confirmation) are disclosed in U.S. Pat. Nos. 10,858,400, 10,017,543, and 9,738,689; US Patent Application Publication Nos. US 2015/0030622, US 2016/0031972, and US 2016/0046675; McLellan et al. (Science (2013) 342:592-598); Krarup et al. (Nature Communications (2015) 6:8143 (Pages 1-12); and Joyce et al. (Nature Structural and Molecular Biology (2016) 23:811-822); the entire contents of each of these references being expressly incorporated herein by reference.

Other non-limiting examples of RSV F protein variants or fragments thereof that can be utilized in accordance with the present disclosure include RSV F protein variants or fragments thereof that include at least one, at least two, at least three, or all four of the amino acid substitutions S155C, 5190F, V207L, and S290C when compared to the native RSV F protein sequence. Particular (but non-limiting) examples of RSV protein variants/fragments that are stabilized in the pre-fusion conformation are disclosed in US 2019/0224300, the entire contents of which are hereby expressly incorporated herein by reference. Other non-limiting examples of RSV F protein variants or fragments thereof that can be utilized in accordance with the present disclosure include prefusion F monomers.

In certain non-limiting embodiments, the RSV F protein variant or fragment thereof is absent a portion or all of a cytoplasmic tail and/or a portion or all of a transmembrane domain of the native RSV F protein. Alternatively, the RSV F protein variant or fragment thereof may include a portion or all of the cytoplasmic tail and/or a portion or all of the transmembrane domain of the native RSV F protein. Particular (but non-limiting) examples of RSV protein variants/fragments that are absent a portion or all of a cytoplasmic tail and/or a portion or all of a transmembrane domain of the native RSV F protein are disclosed in US 2019/0224300, the entire contents of which are hereby expressly incorporated herein by reference.

Another non-limiting example of an RSV F protein that may be utilized in accordance with the present disclosure is an RSV F protein/variant thereof/fragment thereof, including the Fstem fragment, in which the stem region of the RSV F protein/variant/fragment has mutations in amino acids 5573 and N574.

Another non-limiting example of an RSV structural protein that may be present in the RSV-based VLPs is an RSV M2-1 protein or variant or fragment thereof.

In certain particular (but non-limiting) embodiments, the RSV-based VLP comprises at least one amino acid sequence selected from SEQ ID NOs:5, 7, 9, 11, 13, 15, 17, 19, and 21.

In certain particular (but non-limiting) embodiments, the RSV-based VLP comprises at least one non-RSV component. For example (but not by way of limitation), the RSV-based VLP may comprise at least one foreign antigen (such as, but not limited to, an antigen from another virus), thereby allowing the VLP to induce an immune response against both RSV as well as the foreign antigen. Non-limiting examples of foreign antigens that may be utilized in accordance with the present disclosure include an antigen from SARS-CoV-2 (such as, but not limited to, at least a portion of a spike protein, membrane protein, envelope protein, and/or nucleocapsid protein) and/or an antigen from influenza (such as, but not limited to, at least a portion of a hemagglutinin (HA) protein and/or a neuraminidase (NA) protein).

Certain non-limiting embodiments of the present disclosure are directed to an isolated nucleotide sequence that comprises one or more of SEQ ID NOs:4, 6, 8, 10, 12, 14, 16, 18, and 20. In certain particular (but non-limiting) embodiments, the isolated nucleotide sequence encodes an RSV-based VLP as described in detail herein above.

Certain non-limiting embodiments of the present disclosure are also directed to an isolated immunogenic composition comprising one or more of any of the virus-like particles described or otherwise contemplated herein.

In other particular (but non-limiting) embodiments, the immunogenic compositions further include at least one additional RSV-based virus-like particle, wherein the additional RSV-based VLP comprises: an RSV phosphoprotein (P) or variant or fragment thereof; an RSV matrix (M) protein or variant or fragment thereof; and an RSV fusion (F) protein variant or fragment thereof, wherein the RSV F protein variant or fragment thereof contains at least one mutation that stabilizes the F protein in pre-fusion form.

Further non-limiting embodiments of the present disclosure are directed to a pharmaceutical composition that includes a therapeutically effective amount of one or more of any of the RSV-based virus-like particles described in detail herein above or otherwise contemplated herein. In certain non-limiting embodiments, the pharmaceutical composition is capable of eliciting an immune response against the virus or a component thereof in a mammal. In particular (but non-limiting) embodiments, the therapeutically effective amount of the one or more virus-like particles is further defined as an amount sufficient to induce an immune response protective against RSV infection. Thus, in a particular (but non-limiting) embodiment, the pharmaceutical composition may be an immunogenic composition, such as (but not limited to) a vaccine.

In certain non-limiting embodiments, the pharmaceutical composition may further include at least one additional RSV-based virus-like particle, wherein the additional RSV-based VLP comprises: an RSV phosphoprotein (P) or variant or fragment thereof; an RSV matrix (M) protein or variant or fragment thereof; and an RSV fusion (F) protein variant or fragment thereof, wherein the RSV F protein variant or fragment thereof contains at least one mutation that stabilizes the F protein in pre-fusion form.

The pharmaceutical compositions or formulations disclosed or otherwise contemplated herein include one or more virus-like particles as described herein, each of which is substantially purified and/or isolated, except that one or more of such virus-like particles may be included in a single composition. In certain non-limiting embodiments, the pharmaceutical compositions also include a pharmaceutically acceptable carrier or excipient. Any carriers or excipients known in the art may be utilized in accordance with the present disclosure. For example (but not by way of limitation), a physiological compatible carrier (e.g., saline) that is compatible with maintaining the structure/activity of the virus-like particles when administered, and compatible with the desired mode of administration, may be utilized as the pharmaceutically acceptable carrier in accordance with the present disclosure. In addition, the active ingredients may be mixed with excipients which are pharmaceutically acceptable and compatible with the active ingredients. Suitable excipients include, for example but not by way of limitation, water, saline, dextrose, glycerol, ethanol, and the like, or any combination thereof.

The preparation of such compositions for use as immunogenic compositions, such as (but not limited to) vaccines, is well known to those of skill in the art. Typically, such compositions are prepared either as liquid solutions or suspensions; however, solid forms such as (but not limited to) tablets, pills, powders, and the like are also contemplated. Solid forms suitable for solution in, or suspension in, liquids prior to administration may also be prepared. The preparation may also be emulsified. In addition, the pharmaceutical compositions disclosed or otherwise contemplated herein may contain minor amounts of auxiliary substances, such as (but not limited to) wetting or emulsifying agents, pH buffering agents, and the like, as well as any combination thereof. If it is desired to administer an oral form of the pharmaceutical composition, one or more of various thickeners, flavorings, diluents, emulsifiers, dispersing aids, binders, or the like, as well as any combination thereof, may be added. The pharmaceutical compositions of the present disclosure may contain any such additional ingredients so as to provide the composition in a form suitable for administration.

In addition, in certain non-limiting embodiments, the pharmaceutical composition does not contain any adjuvants; rather, the RSV-based VLPs are self-adjuvanting and do not require the presence of additional substance to function as an adjuvant.

In other non-limiting embodiments, the pharmaceutical composition may contain one or more adjuvants known in the art.

The virus-like particles may be present in the pharmaceutical composition at any percentage of concentration that allows the virus-like particles to function as described or as otherwise contemplated herein. For example (but not by way of limitation), the virus-like particles may be present in a sufficient amount to function as an immunogenic composition. In certain particular (but non-limiting) embodiments, the virus-like particles are present in the pharmaceutical composition at a percent concentration of about 0.001%, about 0.005%, about 0.01%, about 0.05%, about 0.1%, about 0.5%, about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 99%. In addition, the scope of the presently disclosure also includes the presence of the virus-like particle in the pharmaceutical composition at any percent concentration that falls within any range formed from the combination of two values listed above (for example, a range of from about 1% to about 99%, a range of from about 2% to about 80%, a range of from about 3% to about 60%, a range of from about 10% to about 95%, a range of from about 40% to about 75%, etc.).

Likewise, a pharmaceutically acceptable carrier and/or excipient may be present in the pharmaceutical composition at any percentage of concentration that allows the carrier/excipient to function as described or as otherwise contemplated herein. In certain particular (but non-limiting) embodiments, each of the pharmaceutically acceptable carrier and/or excipient is present in the pharmaceutical composition at a percent concentration of about 0.001%, about 0.005%, about 0.01%, about 0.05%, about 0.1%, about 0.5%, about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 99%. In addition, the scope of the presently disclosure also includes the presence of each of the pharmaceutically acceptable carrier and/or excipient in the pharmaceutical composition at any percent concentration that falls within any range formed from the combination of two values listed above (for example, a range of from about 1% to about 99%, a range of from about 2% to about 80%, a range of from about 3% to about 60%, a range of from about 10% to about 95%, a range of from about 40% to about 75%, etc.).

The pharmaceutical compositions of the present disclosure may be administered by any of the many suitable means described herein and/or which are well known to those of skill in the art, including but not limited to: by inhalation, intrapulmonary, intranasal, oral, injection (such as, but not limited to, intramuscular, intraperitoneal, intravitreal, or intravenous), or intradermal administration; by ingestion of a food or probiotic product containing the virus; by topical administration, such as (but not limited to) as eye drops, sprays, etc.; and the like. In one instance, the administration will be carried out by using an implant.

In particular (but non-limiting) embodiments, the mode of administration is by intranasal, intrapulmonary, and/or inhalation. For example (but not by way of limitation), nebulized aerosols may be delivered via inhalation using any convention inhaler or nebulizer devices known in the art or otherwise contemplated herein.

One or more than one route of administration can be employed either simultaneously or partially or wholly sequentially, i.e., prime boost vaccine regimens are also contemplated. Such prime boost vaccine regimens typically involve repeated vaccine administration at preselected intervals, such as (but not limited to) at 1 month or 6 weeks of age then at 6 months, 1 year, and yearly thereafter, or at longer intervals, e.g., every 5 or 10 years, etc. Those of skill in the art are well acquainted with the planning, implementation, and assessment of such vaccine strategies, and therefore no further discussion thereof is required.

The pharmaceutical compositions may be administered in conjunction with other pharmaceutical compositions containing other VLPs constructed in accordance with the present disclosure and/or other treatment modalities. In some embodiments, such additional treatment modalities may include (but are not limited to) various substances that boost the immune system, various chemotherapeutic agents, vitamins, anti-allergy agents, anti-inflammatory agents, etc. In other embodiments, other antigenic agents (e.g., other vaccines or vaccinogens), may be advantageously administered or co-administered with the pharmaceutical compositions disclosed or otherwise contemplated herein. For example, in some cases it may be desirable to combine any of the recombinant virus pharmaceutical compositions disclosed or otherwise contemplated herein with other known vaccines which induce protective responses to other agents, particularly other childhood viruses or other infectious agents. The other vaccines may be live attenuated virus vaccines, but this need not always be the case; such vaccines may be inactivated virus vaccines or vaccines against other etiological agents (e.g., bacteria). When multiple immunogenic compositions/vaccines are to be administered together, the immunogenic compositions/vaccine agents may be combined in a single pharmaceutical composition. Alternatively (and/or in addition thereto), the multiple immunogenic compositions/vaccines may be administered separately but over a short time interval, e.g., at a single visit at a doctor's office or clinic, etc.

Certain non-limiting embodiments of the present disclosure are directed to polynucleotides that encode the various components of one or more of any of the RSV-based VLPs disclosed or otherwise contemplated herein. For example (but not by way of limitation), the polynucleotides of the present disclosure may comprise: (a) a gene encoding an RSV phosphoprotein (P) or variant or fragment thereof; (b) a gene encoding an RSV matrix (M) protein or variant or fragment thereof; and (c) a gene encoding an RSV attachment glycoprotein (G) or variant or fragment thereof, wherein the G protein or variant or fragment thereof comprises SEQ ID NO:1 or comprises SEQ ID NOS:1 and 2 (14 amino acid CCR and heparin-binding domain, respectively). In certain particular (but non-limiting) embodiments, at least one of the genes (a)-(c) has been codon-optimized.

Certain non-limiting embodiments of the present disclosure are directed to vectors that encode at least a portion of one or more of any of the RSV-based VLPs disclosed or otherwise contemplated herein. For example (but not by way of limitation), the vectors of the present disclosure may comprise: (a) a polynucleotide encoding an RSV phosphoprotein (P) or variant or fragment thereof; (b) a polynucleotide encoding an RSV matrix (M) protein or variant or fragment thereof; and (c) a polynucleotide encoding an RSV attachment glycoprotein (G) or variant or fragment thereof, wherein the G protein or variant or fragment thereof comprises SEQ ID NO:1 or comprises SEQ ID NOS:1 and 2 (14 amino acid CCR and heparin-binding domain, respectively). In certain particular (but non-limiting) embodiments, at least one of the polynucleotides (a)-(c) has been codon-optimized.

In still yet another aspect, mammalian cells or mammals are provided which include one or more of any of the virus-like particles as described or otherwise contemplated herein, or which include polynucleotide(s) that encode all of the various components of one or more of any of the virus-like particles, as described or otherwise contemplated herein.

In particular, certain non-limiting embodiments of the present disclosure are directed to at least one cell that is capable of producing one or more of any of the virus-like particles described or otherwise contemplated herein. For example, certain non-limiting embodiments of the present disclosure include a mammalian cell that has been transfected with one or more of any of the polynucleotides and/or vectors described or otherwise contemplated herein such that the cell produces one or more of any of the RSV-based virus-like particles described or otherwise contemplated herein.

Any cell type capable of producing one or more of the virus-like particles and capable of functioning as described or otherwise contemplated herein falls within the scope of the present disclosure. In certain non-limiting embodiments, the cell is a mammalian cell. In particular (but non-limiting) embodiments, the mammalian cell is a 293 cell.

Yet further non-limiting embodiments of the present disclosure are directed to a method of producing one or more of any of the virus-like particles described or otherwise contemplated herein. In one non-limiting embodiment of the method, a cell line that expresses one or more of any of the RSV-based VLPs disclosed or otherwise contemplated herein is provided and cultured under conditions that allow for production of the one or more virus-like particles, and then the one or more RSV-based virus-like particles are recovered. In certain particular (but non-limiting) embodiments, the one or more virus-like particles are isolated away from the cultured cells. In a particular (but non-limiting) embodiment, the one or more virus-like particles is substantially purified.

Yet further non-limiting embodiments of the present disclosure are directed to a use of one or more of any of the virus-like particles disclosed or otherwise contemplated herein for the manufacture of a medication for eliciting an immune response in a mammal. In a particular (but non-limiting) embodiment, the medication so produced is a vaccine.

Yet further non-limiting embodiments of the present disclosure are directed to a method of eliciting an immune response in a subject. In the method, one or more of any of the pharmaceutical compositions disclosed or otherwise contemplated herein is introduced into the subject. When two or more of the pharmaceutical compositions are introduced, the two or more pharmaceutical compositions may be administered simultaneously or wholly or partially sequentially.

Certain additional non-limiting embodiments of the present disclosure are directed to a method of generating antibodies specific for RSV in a subject. In the method, one or more of any of the virus-like particles disclosed or otherwise contemplated herein (or one or more of any of the pharmaceutical compositions containing same, as disclosed or otherwise contemplated herein) is introduced into the subject. Antibodies which specifically recognize one of the proteins or fragments thereof present in the virus-like particles may be used to detect production of the particular protein(s)/fragment(s), either in a laboratory setting (e.g., for research purposes) and/or to monitor infections established with the virus-like particles in a subject. Antibodies which specifically recognize the virus-like particles disclosed or otherwise contemplated herein (both mono- and polyclonal) are also encompassed by the present disclosure. In some embodiments, antibody recognition is selective rather than specific. Antibodies may be polyclonal or monoclonal.

Certain additional non-limiting embodiments of the present disclosure are directed to a method of preventing or reducing the occurrence or severity of respiratory syncytial virus infection in a subject. In the method, one or more of any of the pharmaceutical compositions disclosed or otherwise contemplated herein is administered to the subject.

The amount of virus-like particles that is administered to a subject in need thereof varies according to many factors, e.g., the age, weight, overall health, gender, genetic history, history of allergies, prior infection, or vaccine history, etc. of the subject. The pharmaceutical compositions can be administered in a manner compatible with the dosage formulation and in such amounts as will be therapeutically effective (e.g., immunogenic and/or protective against infection with a wild type virus). The quantity to be administered depends on the subject to be treated, including, for example, the capacity of the immune system of the individual to synthesize antibodies, and, if needed, to produce a cell-mediated immune response. Precise amounts of active ingredient required to be administered depend on the judgment of the practitioner and may be monitored on a patient-by-patient basis. The dosage may also depend, without limitation, on the route of administration, the patient's state of health and weight, and the nature of the formulation.

Upon inoculation with the one or more pharmaceutical/vaccine compositions disclosed or otherwise contemplated herein, the immune system of the host can respond to the vaccine by producing antibodies, both secretory and serum, specific for the epitope(s) included in the virus-like particles. As a result of the vaccination, the host can become partially or completely immune to RSV infection, or to developing moderate or severe RSV infection, particularly of the lower respiratory tract. The immune response may be innate or adaptive, and may be either cell-mediated or humoral. In a particular (but non-limiting) embodiment, the response is adaptive and leads to immunological memory. In a particular (but non-limiting) embodiment, the response is protective, i.e., the response prevents or at least lessens the impact of (e.g., avoids development of serious symptoms of) infection by other viruses with shared antigens and/or epitopes, e.g., other Pneumoviridae such as (but not limited to) wild type Pneumoviridae. Single or multiple administrations of the one or more pharmaceutical compositions disclosed or otherwise contemplated herein can be carried out in any of the methods disclosed herein. In neonates and infants, multiple administrations may be required to elicit sufficient levels of immunity. Administration can begin within the first month of life and continue at intervals throughout childhood, such as (but not limited to) at two months, six months, one year, and two years, as necessary to maintain sufficient levels of protection against the pathogen of interest. Similarly, adults who are particularly susceptible to repeated or serious infection by the pathogen of interest, such as (but not limited to) health care workers, day care workers, the elderly, individuals with compromised immune function, and individuals with compromised cardiopulmonary function, may require multiple immunizations to establish and/or maintain protective immune responses. Levels of induced immunity can be monitored by measuring amounts of neutralizing secretory and serum antibodies, and dosages adjusted and/or vaccinations repeated as necessary to maintain desired levels of protection.

Subjects who may be immunized using the formulations of pharmaceutical compositions disclosed or otherwise contemplated herein are usually mammals and are frequently humans, particularly human infants or children. However, this need not always be the case. Veterinary uses of the pharmaceutical compositions and methods disclosed or otherwise contemplated herein are also contemplated, e.g., for companion pets, ruminants, or other animals that are of commercial value e.g., as a food source, or for any other animal, etc.

In certain embodiments of the methods disclosed herein, the method may include a step of identifying suitable recipients and/or of evaluating or monitoring the patient's reaction or response to administration of the composition(s). In some embodiments, the composition comprises one or more RSV-based virus-like particles (as described herein above or otherwise contemplated herein), and the subject is a child, an immunocompromised individual, an elderly patient, and/or any patient at risk of being exposed to RSV and developing an RSV infection. The method may be a method of vaccinating such individuals against developing severe (or alternatively, moderate) lower respiratory tract disease, e.g., against developing bronchiolitis.

The virus-like particles disclosed or otherwise contemplated herein can also be used in diagnostic applications. In one non-limiting embodiment, a method useful for detecting the presence or absence of an antibody specifically reactive with an epitope is provided. The method includes the steps of contacting a sample with the virus-like particles carrying the epitope, and detecting any binding between an antibody component in the sample and the virus-like particle. Examples of binding assays that are suitable for this purpose include (but are not limited to) ELISA (enzyme-linked immunosorbent assay), RIA (radioimmunoassay), FACS (fluorescence-activated cell sorter), and any combinations thereof.

Certain additional non-limiting embodiments of the present disclosure are directed to a method of preventing or reducing the occurrence or severity of respiratory syncytial virus infection in a mammal by administering one or more of any of the virus-like particles disclosed or otherwise contemplated herein (or any of the pharmaceutical compositions containing same) to a mammal. In certain non-limiting embodiments, the mammal is susceptible to infection with RSV.

The RSV-based VLPs utilized in the methods of the present disclosure are self-adjuvanting and do not require the presence of additional substance to function as an adjuvant. Therefore, in certain non-limiting embodiments, no adjuvants are administered to the mammal in the methods described herein.

In other non-limiting embodiments, the methods of the present disclosure can include the step of administering one or more adjuvants, either simultaneously or wholly or partially sequentially with the RSV-based VLPs of the present disclosure.

The RSV-based VLPs can function as immunogenic compositions and vaccines and thus be administered alone for the generation of antibodies and immune responses thereto; thus, in certain non-limiting embodiments, the RSV-based VLPs disclosed or otherwise contemplated herein are utilized alone. Alternatively, other non-limiting embodiments of the present disclosure involve the use of two or more RSV-based VLPs in combination with each other. As such, certain non-limiting embodiments of the present disclosure are directed to a kit that comprises two or more of any of the RSV-based VLPs disclosed or otherwise contemplated herein (or two or more of the pharmaceutical compositions containing same). When two RSV-based VLPs are introduced or administered to a subject in any of the methods taught herein, they may be administered simultaneously or wholly or partially sequentially.

Another alternative involves the use of one or more of any of the RSV-based VLPs described or otherwise contemplated herein in combination with at least one additional RSV-based virus-like particle, wherein the additional RSV-based VLP comprises: an RSV phosphoprotein (P) or variant or fragment thereof; an RSV matrix (M) protein or variant or fragment thereof; and an RSV fusion (F) protein variant or fragment thereof, wherein the RSV F protein variant or fragment thereof contains at least one mutation that stabilizes the F protein in pre-fusion form. As such, certain non-limiting embodiments of the present disclosure are directed to a kit that comprises one or more of any of the RSV-based VLPs disclosed or otherwise contemplated herein (or one or more of the pharmaceutical compositions containing same) in combination with this particular additional RSV-based VLP (or a pharmaceutical composition containing same). When these two RSV-based VLPs are introduced or administered to a subject in any of the methods taught herein, they may be administered simultaneously or wholly or partially sequentially.

In yet another alternative, other non-limiting embodiments of the present disclosure involve the use of one or more RSV-based VLPs in combination with a live RSV vaccine in a prime-boost immunization protocol. As such, certain non-limiting embodiments of the present disclosure are directed to a kit that comprises one or more of any of the RSV-based VLPs disclosed or otherwise contemplated herein (or one or more of any of the pharmaceutical compositions containing same) in combination with a live, attenuated respiratory syncytial virus (RSV).

Any live, attenuated RSV's known in the art or otherwise contemplated herein may be utilized in accordance with the present disclosure. One particular (but non-limiting) example of a live, attenuated RSV that may be utilized in accordance with the present disclosure is a recombinant RSV lacking a gene that encodes a matrix (M) protein of the RSV (RSV M-null). M-null live, attenuated viruses that can be utilized in accordance with the present disclosure are disclosed in detail in International Patent Application Publication No. WO 2012/167139 and issued U.S. Pat. No. 10,844,357; the entire contents of which are hereby expressly incorporated herein by reference in their entirety.

Other non-limiting examples of live, attenuated RSVs that can be utilized in accordance with the present disclosure include those disclosed in detail in U.S. Pat. No. 10,799,576; the entire contents of each of which are hereby expressly incorporated herein by reference.

In certain non-limiting embodiments, the live, attenuated virus is capable of infecting a cell in a mammal but cannot transmit from said cell to another cell in the mammal.

Therefore, any of the methods disclosed or otherwise contemplated herein may comprise the additional step of administering to the subject any of the live, attenuated respiratory syncytial viruses disclosed or otherwise contemplated herein.

In particular (but non-limiting) embodiments, the live, attenuated virus is administered to the mammal prior to the pharmaceutical composition, so that the live virus serves as the “prime” in the prime-boost regimen, while the later-administered RSV-based VLPs serve as the “boost.”

### EXAMPLES

Examples are provided hereinbelow. However, the present disclosure is to be understood to not be limited in its application to the specific experimentation, results, and laboratory procedures disclosed herein. Rather, the Examples are simply provided as one of various embodiments and are meant to be exemplary, not exhaustive.

Example 1—Summary of Design of Various RSV-VLP Constructs

RSV is the single largest viral cause of pediatric bronchiolitis and pneumonia and results in >100,000 deaths each year globally and up to 150,000 hospitalizations in the US in children less than 5 years of age. Despite being a single serotype, RSV re-infects individuals because replicating RSV dysregulates the host response and interferes with induction of adequate immune memory. As a consequence, RSV is also a serious health concern in the elderly and immunocompromised population. A vaccine trial in the 1960s with formaldehyde-inactivated adjuvanted vaccine failed to protect and instead resulted in virus-enhance lung disease (VED) upon subsequent natural RSV exposure. Since then, distinct live and non-live vaccine approaches have been developed and tested, but these approaches have shown that it is exceedingly difficult to produce a vaccine that is both sufficiently efficacious and safe. Due to the special needs of health-compromised individuals (congenital diseases, asthma, transplant patients, etc.) and the immature or waning immune system of the very young and old, respectively, it is believed that more than one type of vaccine may be required to meet the vaccine needs.

Among non-live approaches, virus-like particles (VLPs) are gaining traction. VLPs are particles that include certain structural proteins from a virus but do not contain any genetic material from the virus (and thus cannot cause an infection). VLPs do not replicate yet can accurately mimic the natural virus and induce relevant immune responses. Unlike protein-subunit approaches, which in many cases induced undesirable VED-like symptoms and failed to induce an appropriate cellular response, VLPs in general can cross-present to induce both Th1 and Th2 responses without adjuvants. Recent RSV reports confirm that VLPs without adjuvant can induce protective anti-RSV immunity without VED (Lee et al., Vaccine (2014) 32:5866-5874; Lee et al., Virology (2015) 476:217-225); McGinnes et al., J Virol (2015) doi:10.1128/jvi.00384-15; and Schmidt et al., J Virol (2014) 88:10165-10176). These vaccines also show improved memory relative to live RSV presumably due to the absence of immune dysregulation by de novo synthesized RSV proteins.

VLP approaches currently in preclinical trials are based on heterologous VLP systems, with most expressing the RSV fusion (F) protein, one of two major RSV glycoproteins. It is now well recognized that the F protein is unstable and readily shifts to a post-fusion (non-functional) conformation during purification or vaccine preparation. In recent developments, a pre-fusion stabilized F form (preF) was formulated, and was shown to induce a higher proportion of RSV-neutralizing antibodies than wildtype F (McLellan et al., 2013), making preF an important vaccine antigen. However, a large body of work shows that other RSV antigens including attachment protein G and nucleocapsid protein N can also significantly contribute to protection. Furthermore, even a successful single-antigen vaccine can induce resistant viruses in the population, as demonstrated with Palivizumab studies in cotton rats and in humans (Zhu et al., J Inf Dis (2012) 205:635-638). To broaden and enhance efficacy and simultaneously overcome dependence on a singular antigen, authentic RSV-based VLPs were developed in this Example that are morphologically indistinguishable from wildtype RSV. In particular (but not by way of limitation), VLPs separately displaying the preF protein or the central conserved region of the attachment protein G (G-CCR) were generated separately. The G-CCR harbors a receptor binding domain, and anti-G-CCR Abs can independently neutralize divergent RSV strains and reduce lung pathology. Thus, certain non-limiting embodiments of the present disclosure provide a vaccination strategy whereby combinations of authentic RSV VLPs, separately displaying the preF protein or the G-CCR, are combined. By using distinct VLPs, an optimal ratio of F to G immunogenicity can be determined. Additionally, these VLPs include conserved core RSV antigens for which antibodies or CD8 T cell responses were observed in humans. Relative to an F-alone approach, this strategy thus further enhances efficacy and cross-protection potential, while reducing the likelihood of escape viruses and of immune dysregulation as seen with live RSV.

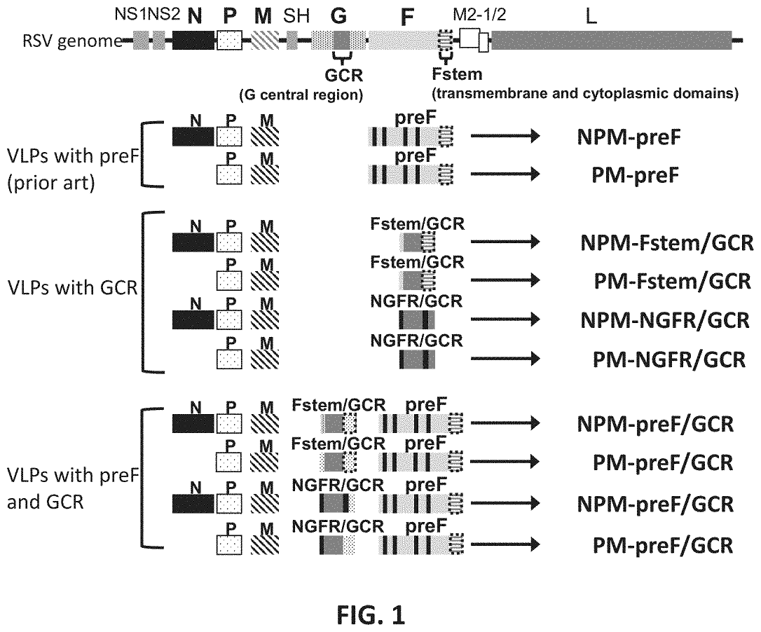

FIG. 1 provides a map of various RSV-based virus-like particles (RSV-VLPs) constructed in accordance with the present disclosure. VLPs with the F protein stabilized in the pre-fusion form have been disclosed (see, for example, Meshram et al. (J Virol (2016) 90(23):10612-10628) and Meshram et al. (Virology (2019) 532:48-54)). VLPs with similar types of structures are labeled “VLPs with preF.”

Non-limiting examples of RSV-based VLPs constructed in accordance with the present disclosure include those labeled as “VLPs with GCR” and “VLPs with preF and GCR.” These two groups of RSV-based VLPs are discussed in detail herein below. Each of these groups contains, at a minimum, the RSV P and M structural proteins in combination with the RSV G structural protein or a fragment thereof that contains a portion of the G protein referred to as the GCR, as discussed in detail below.

Each of the RSV-based VLPs constructed in accordance with the present disclosure include at least a portion of the G protein referred to herein as the G central region (GCR). The GCR includes a highly conserved 14 amino acid sequence of G which is often referred to as the central-conserved-region (CCR). This CCR contains the important CX₃C receptor binding domain. The GCR region (G amino acids 137-211) includes CCR but is larger and includes a heparin-binding-domain in G, which the virus also uses to attach to cells.

In certain non-limiting embodiments, the GCR has an arbitrary length of 75 amino acids; however, this length can be made shorter or longer to further regulate the immune response. As disclosed in further detail in Example 2 below, many different lengths of the GCR were screened for surface expression levels and recognition by known antibodies. While many were found to work, the fragment that included residues 137-211 of G protein was among the best.

In certain non-limiting embodiments, the RSV-VLPs include the entire RSV G protein. Alternatively, the RSV-VLPs may only include the GCR. GCR just by itself cannot be expressed at the cell surface, as it lacks a transmembrane domain (i.e., signal peptide and membrane anchor). Therefore, either the full-length G protein must be used, or the GCR must be fused to a “stem” portion of another protein to obtain efficient expression of the GCR at the surface of the VLP. For example, GCR was fused to the stem of the F protein, and the Fstem-GCR fusion was efficiently expressed at the cell surface and incorporated into RSV based particles for vaccine purposes. In another non-limiting example, GCR was fused to the stem of human NGFR (nerve growth factor receptor, a human surface protein), and the NGFR-GCR fusion maintains the F cytoplasmic domain necessary for incorporation into the particle. Likewise, additional stem/stalk regions from other transmembrane proteins can be used to improve the vaccine.

In addition to the P and M proteins and the GCR fusion, the RSV-VLPs of the present disclosure may include or may be produced in the absence of the RSV N protein. Both RSV-VLPs with and without N work as vaccines. Because N binds RNA and will also bind host RNA, VLPs without N will likely have less host DNA (considered a contamination or impurity) in the vaccine, which could be an advantage. Vaccines with N will probably also generate anti-N Abs, which contribute to immunity.

Further, additional viral sequences can also be incorporated into GCR-expressing genes, to further increase or broaden the immune response.

In a non-limiting example, a preF variant utilized in accordance with the present disclosure is an F gene in which stabilizing mutations based on DS-Cav1 were introduced (McLellan et al., Science (2013) 342:592-598); mutations that stabilize DS-Cav1 are well known in the art. However, any other stabilized F protein or derivative can also be utilized in accordance with the present disclosure to improve the vaccine or focus the response on particular regions of F.

Particular nucleotide and amino acid sequences utilized in this Example are provided herein below. It will be understood that certain non-limiting embodiments of the present disclosure are directed to RSV-VLPs that include one or more of any of SEQ ID NOS:5, 7, 9, 11, 13, 15, 17, 19, and/or 21. It will also be understood that certain non-limiting embodiments of the present disclosure are directed to an isolated nucleotide sequence (such as, but not limited to, an isolated nucleotide sequence that encodes an RSV-VLP or portion thereof) that includes one or more of any of SEQ ID NOS:4, 6, 8, 10, 12, 14, 16, 18, and/or 20. However, the below sequences are provided for purposes of illustration only and should not be construed as limiting in any manner to the scope of the present disclosure.

Plasmids expressing the following nucleotide sequences were used for VLP production:

| | |
|---|---|
| N | Nucleocapsid protein; |
| N/flag | Nucleocapsid with N-terminal flag tag; |
| P | Phosphoprotein; |
| P-mut | Phosphoprotein with alanine mutations that enhance production; |
| P/flag-mut | Phosphoprotein with N-terminal flag tag; |
| M | Matrix protein; |
| preF | Fusion protein with stabilizing mutations published by McLellan et al. |
| | (2013); |
| Fstem-GCR | G central region fused to Fstem region; and |
| NGFR-GCR | G central region fused to NGFRstem region (NGFR = nerve growth |
| | factor receptor). |

N sequence (SEQ ID NO:4)—this sequence was codon-optimized for enhanced expression in mammalian cells:

ATGGCCCTGAGCAAAGTGAAGCTGAACGACACCCTGAACAAGGACCAGCTGCTGTCCTCCAGCAAGTACAC
CATCCAGCGCAGCACCGGCGACAGCATCGACACCCCCAACTACGACGTGCAGAAGCACATCAACAAGCTGT
GCGGCATGCTGCTGATCACCGAGGACGCCAACCACAAGTTCACCGGCCTGATCGGCATGCTGTACGCCATG
TCCCGCCTGGGCCGCGAGGACACCATCAAGATCCTGCGCGACGCCGGCTACCACGTGAAGGCCAACGGCG
TGGACGTGACCACCCACCGCCAAGACATCAACGGCAAGGAGATGAAGTTCGAAGTGCTGACCCTGGCCAG
CCTGACCACCGAGATCCAGATCAACATCGAGATCGAGAGCCGCAAGAGCTACAAGAAGATGCTGAAGGAG
ATGGGCGAAGTGGCCCCCGAGTACCGCCACGACAGCCCCGACTGCGGCATGATCATCCTGTGCATCGCCGC
CCTGGTGATCACCAAGCTGGCCGCCGGCGACCGCAGCGGCCTGACCGCCGTGATCCGCCGCGCCAACAAC
GTGCTGAAGAACGAGATGAAGCGCTACAAGGGCCTGCTGCCCAAGGACATCGCCAACAGCTTCTACGAAG
TGTTCGAGAAGCACCCCCACTTCATCGACGTGTTCGTGCACTTCGGCATCGCCCAGAGCAGCACCCGCGGC
GGCAGCCGCGTGGAGGGCATCTTCGCCGGCCTGTTCATGAACGCCTACGGCGCCGGCCAAGTGATGCTGC
GCTGGGGCGTGCTGGCCAAGAGCGTGAAGAACATCATGCTGGGCCACGCCAGCGTGCAAGCCGAGATGG
AGCAAGTGGTGGAAGTGTACGAGTACGCCCAGAAGCTGGGCGGCGAGGCCGGCTTCTACCACATCCTGAA
CAACCCCAAGGCCAGCCTGCTGAGCCTGACCCAGTTCCCCCACTTCAGCAGCGTGGTGCTGGGAAACGCCG
CCGGCCTGGGCATCATGGGCGAGTACCGCGGCACCCCCCGCAACCAAGACCTGTACGACGCCGCCAAGGC
CTACGCCGAGCAGCTGAAGGAGAACGGCGTGATCAACTACAGCGTGCTGGACCTGACCGCCGAGGAGCTG
GAGGCCATCAAGCACCAGCTGAACCCCAAGGACAACGACGTGGAGCTGTAA
N amino acid sequence (SEQ ID NO: 5):
MALSKVKLNDTLNKDQLLSSSKYTIQRSTGDSIDTPNYDVQKHINKLCGMLLITEDANHKFTGLIGMLYAMSRLG
REDTIKILRDAGYHVKANGVDVTTHRQDINGKEMKFEVLTLASLTTEIQINIEIESRKSYKKMLKEMGEVAPEYRH
DSPDCGMIILCIAALVITKLAAGDRSGLTAVIRRANNVLKNEMKRYKGLLPKDIANSFYEVFEKHPHFIDVFVHFGI
AQSSTRGGSRVEGIFAGLFMNAYGAGQVMLRWGVLAKSVKNIMLGHASVQAEMEQVVEVYEYAQKLGGEAG
FYHILNNPKASLLSLTQFPHFSSVVLGNAAGLGIMGEYRGTPRNQDLYDAAKAYAEQLKENGVINYSVLDLTAEEL
EAIKHQLNPKDNDVEL
N/flag sequence (SEQ ID NO: 6) - this sequence was codon-optimized for enhanced
expression in mammalian cells and has a flag tag at position 2 (underlined):
ATGGACTACAAGGACGACGACGACAAGGCCCTGAGCAAAGTGAAGCTGAACGACACCCTGAACAAGGACC
AGCTGCTGTCCTCCAGCAAGTACACCATCCAGCGCAGCACCGGCGACAGCATCGACACCCCCAACTACGAC
GTGCAGAAGCACATCAACAAGCTGTGCGGCATGCTGCTGATCACCGAGGACGCCAACCACAAGTTCACCGG
CCTGATCGGCATGCTGTACGCCATGTCCCGCCTGGGCCGCGAGGACACCATCAAGATCCTGCGCGACGCCG
GCTACCACGTGAAGGCCAACGGCGTGGACGTGACCACCCACCGCCAAGACATCAACGGCAAGGAGATGAA
GTTCGAAGTGCTGACCCTGGCCAGCCTGACCACCGAGATCCAGATCAACATCGAGATCGAGAGCCGCAAGA
GCTACAAGAAGATGCTGAAGGAGATGGGCGAAGTGGCCCCCGAGTACCGCCACGACAGCCCCGACTGCGG
CATGATCATCCTGTGCATCGCCGCCCTGGTGATCACCAAGCTGGCCGCCGGCGACCGCAGCGGCCTGACCG
CCGTGATCCGCCGCGCCAACAACGTGCTGAAGAACGAGATGAAGCGCTACAAGGGCCTGCTGCCCAAGGA
CATCGCCAACAGCTTCTACGAAGTGTTCGAGAAGCACCCCCACTTCATCGACGTGTTCGTGCACTTCGGCAT
CGCCCAGAGCAGCACCCGCGGCGGCAGCCGCGTGGAGGGCATCTTCGCCGGCCTGTTCATGAACGCCTAC
GGCGCCGGCCAAGTGATGCTGCGCTGGGGCGTGCTGGCCAAGAGCGTGAAGAACATCATGCTGGGCCACG
CCAGCGTGCAAGCCGAGATGGAGCAAGTGGTGGAAGTGTACGAGTACGCCCAGAAGCTGGGCGGCGAGG
CCGGCTTCTACCACATCCTGAACAACCCCAAGGCCAGCCTGCTGAGCCTGACCCAGTTCCCCCACTTCAGCA
GCGTGGTGCTGGGAAACGCCGCCGGCCTGGGCATCATGGGCGAGTACCGCGGCACCCCCCGCAACCAAGA
CCTGTACGACGCCGCCAAGGCCTACGCCGAGCAGCTGAAGGAGAACGGCGTGATCAACTACAGCGTGCTG
GACCTGACCGCCGAGGAGCTGGAGGCCATCAAGCACCAGCTGAACCCCAAGGACAACGACGTGGAGCTGTAA
N/flag amino acid sequence (SEQ ID NO: 7):
MDYKDDDDKALSKVKLNDTLNKDQLLSSSKYTIQRSTGDSIDTPNYDVQKHINKLCGMLLITEDANHKFTGLIGM
LYAMSRLGREDTIKILRDAGYHVKANGVDVTTHRQDINGKEMKFEVLTLASLTTEIQINIEIESRKSYKKMLKEMG
EVAPEYRHDSPDCGMIILCIAALVITKLAAGDRSGLTAVIRRANNVLKNEMKRYKGLLPKDIANSFYEVFEKHPHFI
DVFVHFGIAQSSTRGGSRVEGIFAGLFMNAYGAGQVMLRWGVLAKSVKNIMLGHASVQAEMEQVVEVYEYA
QKLGGEAGFYHILNNPKASLLSLTQFPHFSSVVLGNAAGLGIMGEYRGTPRNQDLYDAAKAYAEQLKENGVINYS
VLDLTAEELEAIKHQLNPKDNDVEL
P sequence (SEQ ID NO: 8) - this sequence was codon-optimized for enhanced expression
in mammalian cells:
ATGGAGAAGTTCGCCCCCGAGTTCCACGGCGAGGACGCCAACAACCGCGCCACCAAGTTCCTGGAGAGCA
TCAAGGGTAAGTTCACCAGCCCCAAGGACCCCAAGAAGAAGGACAGCATCATCAGCGTGAACAGCATCGA
CATCGAAGTGACCAAGGAGAGCCCCATCACCAGCAACAGCACCATCATCAACCCCACCAACGAGACCGACG
ACACCGCCGGCAACAAGCCCAACTACCAGCGCAAGCCCCTGGTGAGCTTCAAGGAGGACCCCACCCCCAGC
GACAACCCCTTCAGCAAGCTCTACAAGGAGACCATCGAGACCTTCGACAACAACGAGGAGGAGAGCAGCT
ACAGCTACGAGGAGATCAACGACCAGACCAACGACAACATCACCGCCCGCCTGGACCGCATCGACGAGAA
GCTGAGCGAGATCCTGGGCATGCTGCACACCCTGGTGGTGGCCAGCGCCGGCCCCACCAGCGCCCGCGAC
GGCATCCGCGACGCTATGGTGGGCCTGCGCGAGGAGATGATCGAGAAGATCCGCACCGAGGCCCTGATGA
CCAACGACCGCCTGGAGGCTATGGCCCGCCTGCGCAACGAGGAGAGCGAGAAGATGGCCAAGGACACCA
GCGACGAAGTGAGCCTGAACCCCACCAGCGAGAAGCTGAACAACCTGCTGGAGGGTAACGACAGCGACAA
CGACCTGAGCCTGGAGGACTTCTAA
P amino acid sequence (SEQ ID NO: 9):
MEKFAPEFHGEDANNRATKFLESIKGKFTSPKDPKKKDSIISVNSIDIEVTKESPITSNSTIINPTNETDDTAGNKPN
YQRKPLVSFKEDPTPSDNPFSKLYKETIETFDNNEEESSYSYEEINDQTNDNITARLDRIDEKLSEILGMLHTLVVAS
AGPTSARDGIRDAMVGLREEMIEKIRTEALMTNDRLEAMARLRNEESEKMAKDTSDEVSLNPTSEKLNNLLEGN
DSDNDLSLEDF
P-mut sequence (SEQ ID NO: 10) - this sequence was codon-optimized for enhanced
expression in mammalian cells, and has amino acids 43 and 54 (lower case) mutated
to alanine to enhance VLP production:
ATGGAGAAGTTCGCCCCCGAGTTCCACGGCGAGGACGCCAACAACCGCGCCACCAAGTTCCTGGAGAGCA
TCAAGGGTAAGTTCACCAGCCCCAAGGACCCCAAGAAGAAGGACAGCATCATCAGCgccAACAGCATCGAC
ATCGAAGTGACCAAGGAGgccCCCATCACCAGCAACAGCACCATCATCAACCCCACCAACGAGACCGACGAC
ACCGCCGGCAACAAGCCCAACTACCAGCGCAAGCCCCTGGTGAGCTTCAAGGAGGACCCCACCCCCAGCGA
CAACCCCTTCAGCAAGCTCTACAAGGAGACCATCGAGACCTTCGACAACAACGAGGAGGAGAGCAGCTACA
GCTACGAGGAGATCAACGACCAGACCAACGACAACATCACCGCCCGCCTGGACCGCATCGACGAGAAGCT
GAGCGAGATCCTGGGCATGCTGCACACCCTGGTGGTGGCCAGCGCCGGCCCCACCAGCGCCCGCGACGGC
ATCCGCGACGCTATGGTGGGCCTGCGCGAGGAGATGATCGAGAAGATCCGCACCGAGGCCCTGATGACCA
ACGACCGCCTGGAGGCTATGGCCCGCCTGCGCAACGAGGAGAGCGAGAAGATGGCCAAGGACACCAGCG
ACGAAGTGAGCCTGAACCCCACCAGCGAGAAGCTGAACAACCTGCTGGAGGGTAACGACAGCGACAACGA
CCTGAGCCTGGAGGACTTCTAA
P-mut amino acid sequence (SEQ ID NO: 11):
MEKFAPEFHGEDANNRATKFLESIKGKFTSPKDPKKKDSIISANSIDIEVTKEAPITSNSTIINPTNETDDTAGNKPN
YQRKPLVSFKEDPTPSDNPFSKLYKETIETFDNNEEESSYSYEEINDQTNDNITARLDRIDEKLSEILGMLHTLVVAS
AGPTSARDGIRDAMVGLREEMIEKIRTEALMTNDRLEAMARLRNEESEKMAKDTSDEVSLNPTSEKLNNLLEGN
DSDNDLSLEDF
P/flag-mut sequence (SEQ ID NO: 12) - this sequence was codon-optimized for enhanced
expression in mammalian cells, and has amino acids 43 and 54 (lower case) mutated to
alanine to enhance VLP production, and has a flag tag at position 241 (underlined):
ATGGAGAAGTTCGCCCCCGAATTCCACGGCGAGGACGCCAACAACCGCGCCACCAAGTTCCTGGAGAGCAT
CAAGGGTAAGTTCACCAGCCCCAAGGACCCCAAGAAGAAGGACAGCATCATCAGCgccAACAGCATCGACA
TCGAAGTGACCAAGGAGgccCCCATCACCAGCAACAGCACCATCATCAACCCCACCAACGAGACCGACGACA
CCGCCGGCAACAAGCCCAACTACCAGCGCAAGCCCCTGGTGAGCTTCAAGGAGGACCCCACCCCCAGCGAC
AACCCCTTCAGCAAGCTCTACAAGGAGACCATCGAGACCTTCGACAACAACGAGGAGGAGAGCAGCTACA
GCTACGAGGAGATCAACGACCAGACCAACGACAACATCACCGCCCGCCTGGACCGCATCGACGAGAAGCT
GAGCGAGATCCTGGGCATGCTGCACACCCTGGTGGTGGCCAGCGCCGGCCCCACCAGCGCCCGCGACGGC
ATCCGCGACGCTATGGTGGGCCTGCGCGAGGAGATGATCGAGAAGATCCGCACCGAGGCCCTGATGACCA
ACGACCGCCTGGAGGCTATGGCCCGCCTGCGCAACGAGGAGAGCGAGAAGATGGCCAAGGACACCAGCG
ACGAAGTGAGCCTGAACCCCACCAGCGAGAAGCTGAACAACCTGCTGGAGGGTAACGACAGCGACAACGA
CCTGAGCCTGGAGGACTACAAGGACGACGACGACAAGTTCTAA
P/flag-mut amino acid sequence (SEQ ID NO: 13):
MEKFAPEFHGEDANNRATKFLESIKGKFTSPKDPKKKDSIISANSIDIEVTKEAPITSNSTIINPTNETDDTAGNKPN
YQRKPLVSFKEDPTPSDNPFSKLYKETIETFDNNEEESSYSYEEINDQTNDNITARLDRIDEKLSEILGMLHTLVVAS
AGPTSARDGIRDAMVGLREEMIEKIRTEALMTNDRLEAMARLRNEESEKMAKDTSDEVSLNPTSEKLNNLLEGN
DSDNDLSLEDYKDDDDKF
M sequence (SEQ ID NO: 14) - this sequence was codon-optimized for enhanced
expression in mammalian cells:
ATGGAGACCTACGTGAACAAGCTGCACGAGGGCAGCACCTACACCGCCGCCGTGCAGTACAACGTGCTGG
AGAAGGACGACGACCCCGCCAGCCTGACCATCTGGGTGCCCATGTTCCAGAGCAGCATGCCCGCCGACCTG
CTGATCAAGGAGCTGGCCAACGTGAACATCCTGGTGAAGCAGATCAGCACCCCCAAGGGGCCTAGCCTGC
GCGTGATGATCAACAGCCGCAGCGCCGTGCTGGCCCAGATGCCCAGCAAGTTCACCATCTGCGCCAACGTG
AGCCTGGACGAGCGCAGCAAGCTGGCCTACGACGTGACCACCCCCTGCGAGATCAAGGCCTGCAGCCTGA
CCTGCCTGAAGAGCAAGAACATGCTGACCACCGTGAAGGACCTGACCATGAAGACCCTGAACCCCACCCAC
GACATCATCGCCCTGTGCGAGTTCGAGAACATCGTGACCAGCAAGAAAGTGATCATCCCCACCTACCTGCG
CAGCATCAGCGTGCGCAACAAGGACCTGAACACCCTGGAGAACATCACCACCACCGAGTTCAAGAACGCCA
TCACCAACGCCAAGATCATCCCCTACAGCGGCCTGCTGCTGGTGATCACCGTGACCGACAACAAGGGCGCC
TTCAAGTACATCAAGCCCCAGAGCCAGTTCATCGTGGACCTGGGCGCCTACCTGGAGAAGGAGAGCATCTA
CTACGTGACCACCAACTGGAAGCACACCGCCACCCGCTTCGCCATCAAGCCTATGGAGGACTAA
M amino acid sequence (SEQ ID NO: 15):
METYVNKLHEGSTYTAAVQYNVLEKDDDPASLTIWVPMFQSSMPADLLIKELANVNILVKQISTPKGPSLRVMIN
SRSAVLAQMPSKFTICANVSLDERSKLAYDVTTPCEIKACSLTCLKSKNMLTTVKDLTMKTLNPTHDIIALCEFENIV
TSKKVIIPTYLRSISVRNKDLNTLENITTTEFKNAITNAKIIPYSGLLLVITVTDNKGAFKYIKPQSQFIVDLGAYLEKESI
YYVTTNWKHTATRFAIKPMED
preF sequence (SEQ ID NO: 16) - this sequence was codon-optimized for enhanced
expression in mammalian cells and has the 2 final amino acids (lower case)
mutated to alanine for enhanced packaging:
ATGGAGCTGCTGATCCTGAAGGCCAACGCCATCACCACCATCCTGACCGCCGTGACCTTCTGCTTCGCCAGC
GGCCAGAACATCACCGAGGAGTTCTACCAGAGCACCTGCAGCGCCGTGAGCAAGGGCTACCTGAGCGCCC
TGCGCACCGGCTGGTACACCAGCGTGATCACCATCGAGCTGAGCAACATCAAGGAGAACAAGTGCAACGG
CACCGACGCCAAAGTGAAGCTGATCAAGCAAGAGCTGGACAAGTACAAGAACGCCGTGACCGAGCTGCAG
CTGCTGACCCAGAGCACCCCCGCCACCAACAACCGGGCCCGCCGCGAGCTGCCCCGCTTCATGAACTACAC
CCTGAACAACGCCAAGAAGACCAACGTGACCCTGAGCAAGAAGCGCAAGCGCCGCTTCCTGGGCTTCCTGC
TGGGCGTGGGCAGCGCCATCGCCAGCGGCGTGGCCGTGTGTAAAGTGCTGCACCTGGAGGGCGAAGTGA
ACAAGATCAAGAGCGCCCTGCTGAGCACCAACAAGGCCGTGGTGAGCCTGAGCAACGGCGTGAGCGTGCT
GACCTTCAAAGTGCTGGACCTGAAGAACTACATCGACAAGCAGCTGCTGCCCATCCTCAACAAGCAGAGCT
GCAGCATCAGCAACATCGAGACCGTGATCGAGTTCCAGCAGAAGAACAACCGCCTGCTGGAGATCACCCGC
GAGTTCAGCGTGAACGCCGGCGTGACCACCCCCGTGAGCACCTACATGCTGACCAACAGCGAGCTGCTGAG
CCTGATCAACGACATGCCCATCACCAACGACCAGAAGAAGCTGATGAGCAACAACGTGCAGATCGTGCGCC
AGCAGAGCTACAGCATCATGTGTATCATCAAGGAGGAAGTGCTGGCCTACGTGGTGCAGCTGCCCCTGTAC
GGCGTGATCGACACCCCCTGCTGGAAGCTGCACACCAGCCCCCTGTGCACCACCAACACCAAGGAGGGCAG
CAACATCTGCCTGACGCGTACCGACCGCGGCTGGTACTGCGACAACGCCGGCAGCGTGAGCTTCTTCCCCC
AAGCCGAGACCTGCAAAGTGCAGAGCAACCGCGTGTTCTGCGACACCATGAACAGCCTGACCCTGCCCAGC
GAAGTGAACCTGTGCAACGTGGACATCTTCAACCCCAAGTACGACTGCAAGATCATGACCAGCAAGACCGA
CGTGAGCAGCAGCGTGATCACCAGCCTGGGCGCCATCGTGAGCTGCTACGGGAAGACCAAGTGCACCGCC
AGCAACAAGAACCGCGGCATCATCAAGACCTTCAGCAACGGCTGCGACTACGTGAGCAACAAGGGCGTGG
ACACCGTGAGCGTGGGGAACACCCTGTACTACGTGAACAAGCAAGAGGGGAAGAGCCTGTACGTGAAGG
GCGAGCCCATCATCAACTTCTACGACCCCCTGGTGTTCCCCAGCGACGAGTTCGACGCCAGCATCAGCCAAG
TGAACGAGAAGATCAACCAGAGTCTGGCCTTCATCCGCAAGAGCGACGAGCTGCTGCACAACGTGAACGC
CGGGAAGAGCACCACCAACATCATGATCACCACCATCATCATCGTGATCATCGTGATCCTGCTGAGCCTGAT
CGCCGTGGGCCTGCTGCTGTACTGCAAGGCCCGCAGCACCCCCGTGACCCTGAGCAAGGACCAGCTGAGC
GGCATCAACAACATCGCCTTCgccgccTAA
preF amino acid sequence (SEQ ID NO: 17):
MELLILKANAITTILTAVTFCFASGQNITEEFYQSTCSAVSKGYLSALRTGWYTSVITIELSNIKENKCNGTDAKVKLI
KQELDKYKNAVTELQLLTQSTPATNNRARRELPRFMNYTLNNAKKTNVTLSKKRKRRFLGFLLGVGSAIASGVAV
CKVLHLEGEVNKIKSALLSTNKAVVSLSNGVSVLTFKVLDLKNYIDKQLLPILNKQSCSISNIETVIEFQQKNNRLLEIT
REFSVNAGVTTPVSTYMLTNSELLSLINDMPITNDQKKLMSNNVQIVRQQSYSIMCIIKEEVLAYVVQLPLYGVID
TPCWKLHTSPLCTTNTKEGSNICLTRTDRGWYCDNAGSVSFFPQAETCKVQSNRVFCDTMNSLTLPSEVNLCNV
DIFNPKYDCKIMTSKTDVSSSVITSLGAIVSCYGKTKCTASNKNRGIIKTFSNGCDYVSNKGVDTVSVGNTLYYVNK
QEGKSLYVKGEPIINFYDPLVFPSDEFDASISQVNEKINQSLAFIRKSDELLHNVNAGKSTTNIMITTIIIVIIVILLSLIA
VGLLLYCKARSTPVTLSKDQLSGINNIAFAA
Fstem-GCR sequence (SEQ ID NO: 18) - this sequence was codon-optimized for enhanced
expression in mammalian cells. G amino acids 137-211 are lowercase/underlined. In the
F sequence, the 2 final amino acids (lower case) are mutated to alanine for enhanced
surface expression and packaging:
ATGGAGCTGCTGATCCTGAAAGTGAACGCCATCACCACCATCCTGACCGCCGTGACCTTCTGCTTCGCCAGC
GGCCAGGCCATCACCGAGGAATTCTACCAGAGCaccaccacccagacccagcccagcaagcccaccaccaagcagcgcca
gaacaagcctcccagcaagcccaacaacgacttccacttcgaagtgttcaacttcgtgccttgcagcatttgcagcaacaaccccacttgttg
ggccatttgcaagcgcatccccaacaagaagcccggaaagaagaccaccaccaagcccaccaagaagcccaccctcaagaccaccAAC
GAGAAGATCACCCAGAGTCTGGCCTTCATCCGCAAGAGCGACGAGCTGCTGCACAACGTGAACGCCGGGA
AGAGCACCACCAACATCATGATCACCACCATCATCATCGTGATCATCGTGATCCTGCTGAGCCTGATCGCCG
TGGGCCTGCTGCTGTACTGCAAGGCCCGCAGCACCCCCGTGACCCTGAGCAAGGACCAGCTGAGCGGCATC
AACAACATCGCCTTCGCCGCCTAA
Fstem-GCR amino acid sequence (SEQ ID NO: 19):
MELLILKVNAITTILTAVTFCFASGQAITEEFYQSTTTQTQPSKPTTKQRQNKPPSKPNNDFHFEVFNFVPCSICSN
NPTCWAICKRIPNKKPGKKTTTKPTKKPTLKTTNEKITQSLAFIRKSDELLHNVNAGKSTTNIMITTIIIVIIVILLSLIAV
GLLLYCKARSTPVTLSKDQLSGINNIAFAA
NGFR-GCR sequence (SEQ ID NO: 20) - this sequence was codon-optimized for enhanced
expression in mammalian cells; G amino acids 137-211 are lowercase/underlined:
ATGGGGGCAGGTGCCACCGGCCGCGCCATGGACGGGCCGCGCCTGCTGCTGTTGCTGCTTCTGGGGGTGT
CCCTTGGAGGTGCCAAGGAGGCAaccaccacccagacccagcccagcaagcccaccaccaagcagcgccagaacaagcctccc
agcaagcccaacaacgacttccacttcgaagtgttcaacttcgtgccttgcagcatttgcagcaacaaccccacttgttgggccatttgcaagc
gcatccccaacaagaagcccggaaagaagaccaccaccaagcccaccaagaagcccaccctcaagaccaccGTGGCAGGTGTGGT
GACCACAGTGATGGGCAGCTCCCAGCCCGTGGTGACCCGAGGCACCACCGACAACCTCATCCCTGTCTATT
GCTCCATCCTGGCTGCTGTGGTTGTGGGTCTTGTGGCCTACATAGCCTTCAAGAGGTGA
NGFR-GCR amino acid sequence (SEQ ID NO: 21):
MGAGATGRAMDGPRLLLLLLLGVSLGGAKEATTTQTQPSKPTTKQRQNKPPSKPNNDFHFEVFNFVPCSICSNN
PTCWAICKRIPNKKPGKKTTTKPTKKPTLKTTVAGVVTTVMGSSQPVVTRGTTDNLIPVYCSILAAVVVGLVAYIAFKR

### Example 2

This Example details the design, generation, and characterization of various RSV-based VLPs constructed in accordance with the present disclosure.

Design of a respiratory syncytial virus VLP displaying the G protein central region (GCR)

The RSV transmembrane glycoproteins G and F are the major protective antigens, and each can induce neutralizing anti-viral antibodies (Abs) after infection with RSV. The F protein is relatively well conserved and is an important target for vaccine development. Due to its metastable structure, F can shift prematurely to its post-function conformation. Ongoing efforts aim to better preserve the pre-function conformation of F to allow for improved Ab-mediated neutralization of infectious virus.

G is poorly conserved between viral strains, except for an approximately 50 amino acid region termed the central conserved region (“CCR” or “CCRG”), which is located in between two large highly glycosylated regions. Within the CCRG is a cysteine-rich site (CWAIC (SEQ ID NO:22) or CX₃C) which was shown to mimic fractalkine, a chemokine known to be involved in leucocyte recruitment. The G protein CX₃C site was found to bind to the fractalkine receptor CX₃CR₁ (Tripp et al., Nature Immunology (2001) 2:732-738), and this interaction is now believed to both modulate downstream immune effects that benefit the virus and serve as a port of entry into host lung cells in vivo (Chirkova et al., J General Virol (2015) 96:2543-2556; and Johnson et al., PLoS Pathog (2015) 11(12):e1005318). The G gene also expresses a secreted version of the G protein (Gsec) from an alternative start codon within the G open reading frame. Gsec was shown to serve as an Ab decoy and to block leucocyte recruitment, presumably through CX₃C—CX₃CR1 interaction. The relative contributions of membrane-anchored G (a structural virion component) and Gsec (secreted from RSV infected cells) to lung pathology is not understood.

Antibodies directed to the CCRG show significant potential in the prevention and treatment of RSV-associated disease: they reduce the rate of infection in vivo (Chirkova et al., 2015; and Johnson et al., 2015), and they also significantly reduce RSV-associated lung pathology. Moreover, the CCRG is highly conserved between strains and is thus a logical target for development of a cross-protective vaccine. However, the CCRG is surrounded by large highly glycosylated domains and represents only a minor proportion of the Ab-inducing viral antigens, and may not induce sufficient levels of antibodies to warrant protection. Enhancing exposure of the CCRG in a vaccine to increase the level of anti-CCRG Abs will be highly beneficial. Hence, this Example explored options to present the GCR in isolation (away from the G and F proteins) to focus the immune response onto this area, and in a manner that resembles wild type RSV (i.e., authentic filamentous RSV VLPs) and allows a high level of antigen incorporation.

The production of wild type virus-resembling VLPs by co-expressing the RSV P, M, and F proteins has been reported previously (Meshram et al., J Virol (2016) 90(23):10612-10628). Similar to the morphology of infectious RSV, these VLPs were filamentous in nature. Aside from the absence of G, the PMF-induced VLPs were indistinguishable from wild type RSV by high resolution SEM. Meshram et al. also demonstrated that for F, only its carboxy-terminus was required: an F peptide consisting of the F protein transmembrane domain (TMD), cytoplasmic tail (CT), and approximately 25 amino acids of the ectodomain, named Fstem, was sufficient for VLP formation. Because Fstem is required for, and structurally participates in, VLP formation, grafting of foreign peptides onto the Fstem led to efficient incorporation of the foreign peptides into the VLP membrane. In contrast to F, the G protein is not required for VLP formation, and the mechanism of incorporation of G into RSV particles is not known.

To take advantage of the structural requirement for Fstem in VLP formation and force incorporation of the G central region (GCR) into RSV VLPs, various lengths of the GCR were grafted onto the Fstem domain, with the aim to generate a particle exclusively displaying the GCR at its surface. This particle would morphologically resemble a wild-type virus, contain the internal P and M proteins, and repetitively display at its surface exclusively the GCR region to elicit a response focused on anti-GCR immunity. Such a GCR presenting particle could be used as a component of a broader immunization strategy aimed at preventing RSV disease, or be examined as a stand-alone vaccine for RSV-experienced individuals. It is, however, important to maintain the structural integrity of the GCR region. In this Example, two separate Abs known to bind the GCCR were used to define a GCR region likely to be structurally preserved when grafted in isolation onto Fstem. This Example describes the design and production of a wt virus-resembling VLP that displays high levels of GCR.

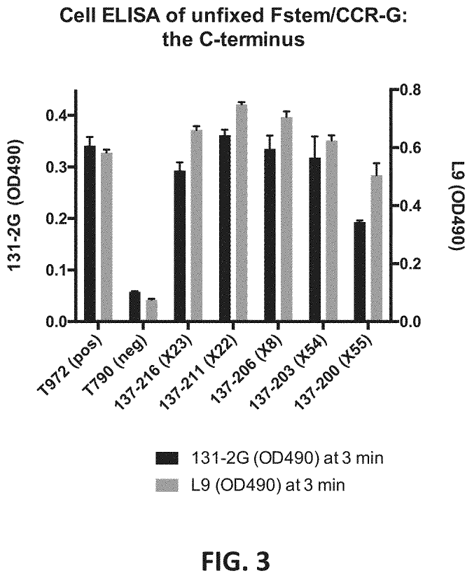

FIGS. 3-5 depict the design of an Fstem-GCR fusion protein that is efficiently expressed at the cell surface (as an indicator of availability for particle assembly) and efficiently recognized by two distinct anti-GCCR Abs (as an indicator of structure preservation).

Since RSV particles are believed to assemble at the plasma membrane, screens were performed for GCR regions that, when grafted onto Fstem, are expressed at the cell surface and recognized by known anti-GCCR Abs 131-2G and L9. An Fstem construct that induced formation of RSV-resembling filamentous VLPs in the presence of P and M was previously reported (Meshram et al., 2016). This construct, previously named FstemAGL, contained a myc tag and lacked the remaining native F glycosylation sites to improve quantitation on western blots. For this Example, the myc tag of FstemAGL was removed, and the resulting base construct was designated Fstem.

To determine the CCRG size optimal for surface expression and structure preservation, the carboxy-terminal end of the CCRG was first varied while keeping the amino-terminal end constant (FIG. 3). Five different CCRG encoding regions ranging from G residues 137-200 to 137-216 were cloned into Fstem, and the resulting Fstem/CCRG plasmids were transfected into HEp-2 cells for cell ELISA analysis. This was done as previously described, with the exception that transfected cells were fixed after incubation with anti-CCRG Abs, instead of before. This was done to avoid structural changes in the GCR region that might result from paraformaldehyde fixation and thus to mimic as best as possible the native GCR structure. A plasmid expressing the intact G protein (containing the CCRG) was used as a positive control. ELISA results using 131-2G and L9 monoclonal Abs showed strong surface recognition only for Fstem/GCR protein with GCR peptides 137-203, 137-206, and 137-211. This suggested that shortening of the GCR beyond residue 203 or lengthening beyond residue 211 negatively impacted either surface transport or structure preservation.

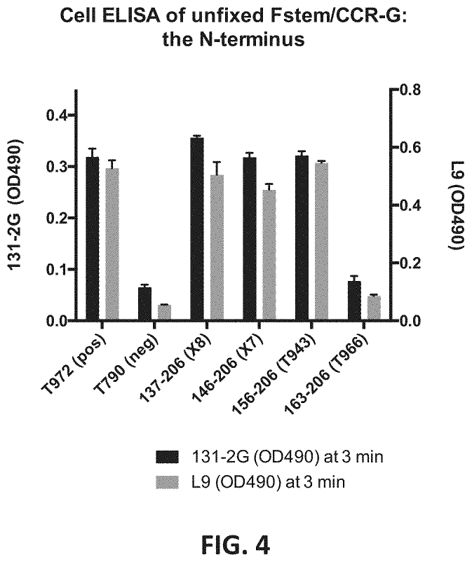

Next, the amino-terminal end of the GCR was varied while keeping the carboxy-terminal end constant, and surface recognition was similarly tested by 131-2G and L9 (FIG. 4). Fstem constructs containing GCR peptides 137-206, 146-206, and 156-206 were efficiently recognized at the cell surface, whereas the 163-206 CCRG was poorly recognized. Thus, deletion of GCR residues past 156 was deleterious for availability at the cell surface or structure preservation. Based on the outcome of FIGS. 3 and 4, the shortest and longest GCR regions to be effectively presented by Fstem and recognized at the cell surface in the native (unfixed) state were defined as residues 156-203 and 137-211.

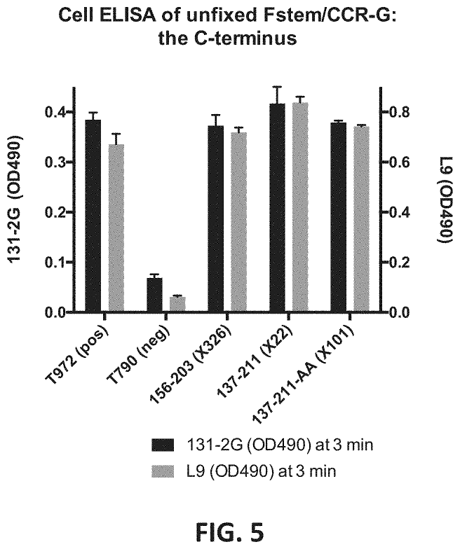

Based on the above, Fstem/GCR proteins with a minimal GCR (156-203) and maximal GCR (137-211) were directly compared (FIG. 5). For Fstem/GCR137-211, a modification in which the two very carboxy-terminal amino acids of Fstem (native F amino acids 5573 and N574) were mutated to alanines was also analyzed. In unpublished observations, this modification appeared to enhance the amount of virus-induced filaments at the cell surface, indicative of increased particle formation or stability. All Fstem/CCRG proteins were readily recognized by anti-CCRG Abs, and Fstem/GCR137-211 provided the strongest ELISA signal.

The above data show that both the short and large GCR peptides were efficiently presented at the cell surface, with Fstem/GCR137-211 providing the strongest signal. Recognition of the native (unfixed) proteins by two distinct anti-CCRG Abs demonstrates that the peptides may be conformationally similar to the GCR region of wildtype G protein.

In FIGS. 3-5, the most optimal GCR peptide length to be efficiently presented at the cell surface when grafted onto Fstem was determined. Next, it was examined whether Fstem/GCR peptides would be incorporated into transiently induced VLPs and which of the Fstem/GCR proteins allowed the most efficient VLP production. Co-expression of P, M, and Fstem proteins was previously shown to induce formation of filamentous VLPs; these VLPs could be visualized by SEM and harvested and quantitated on western blots (Meshram et al., 2016). Using a protocol for induction and harvest described herein below in Example 3, seven distinct Fstem/CCRG constructs were examined. For P, alanine was substituted for valine 43 and serine 54 (V43A, S54A), as this modification was found in preliminary experiments to increase the amount of VLPs (not shown). The P protein was also tagged with a flag epitope at its carboxy terminus, in order to detect P on western blots. The resulting P construct was designated P/flag. Plasmids expressing the different Fstem/GCR proteins were co-transfected with P/flag and M proteins into HEp-2 cells. As positive controls for VLP production, previously established combinations were included (P, M, FstemAGL and P, M, F). As negative controls, Fstem/GCR proteins were expressed in the absence of P/flag and M. At 40, cells were scraped and agitated by pipetting up and down. Cell debris was cleared by low-speed centrifugation, and VLPs were pelleted through a 20% sucrose cushion and dissolved in Laemli buffer. Samples were electrophoresed and western blots generated. Blots were incubated with anti-flag Ab to detect P, anti-M peptide serum, and anti-G Ab 131-2G.

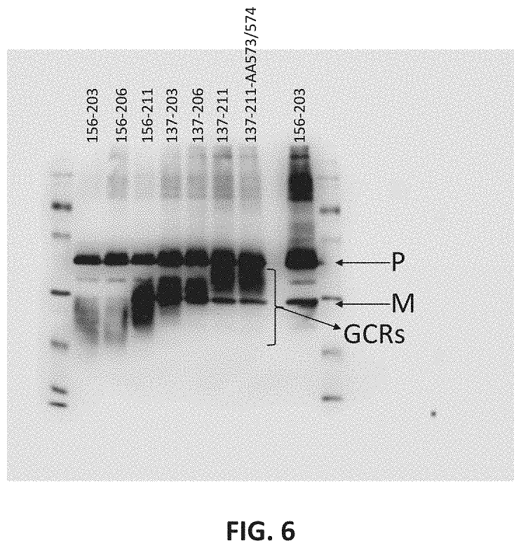

As shown in FIG. 6, expression of Fstem/GCR proteins alone did not yield significant levels of Fstem/GCR protein on western blot, consistent with our previous finding that P, M, and F are each required for efficient particle formation. In contrast, all combinations of co-expressed viral proteins yielded VLPs that contained P/flag, M, and Fstem/GCR. Fstem/GCR proteins varied in molecular weight and were heterogenous in size, which may in part be due to a varied serine/threonine content with potential for O-linked glycosylation (wildtype G protein is highly 0-link glycosylated). Due to the “smeary” nature of the Fstem/GCR protein bands, quantitation could not be accurately performed. However, the combination of P/flag, M, and Fstem/CCRG137-211/5573A-N574A consistently showed the highest VLP yield. Therefore, this combination was used to visually examine VLPs at high resolution (EM).

Based on the ability of Fstem/GCR with G amino acids 137-211-S573A/N574A to be expressed well and effectively recognized by two distinct CCRG Abs in a native (unfixed) cell ELISA, this construct was selected as the initial Fstem/CCRG candidate to target the CCRG to VLPs.

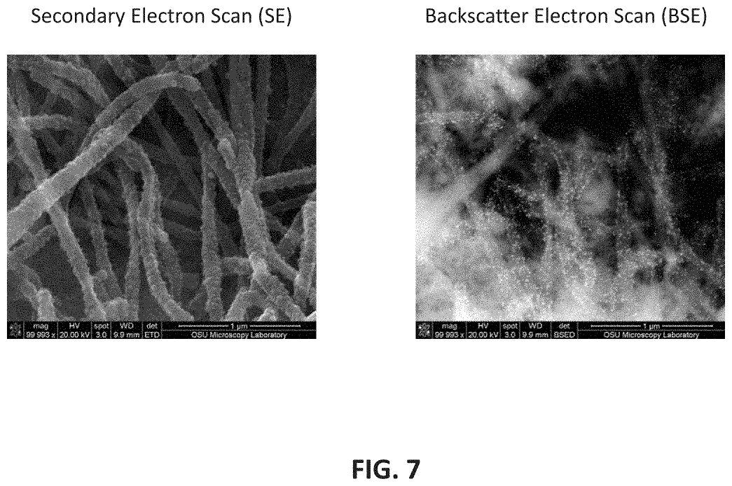

VLPs were generated in HEP-2 cells as described above, fixed, and stained for scanning EM. Samples were also incubated with anti-GCCR Ab L9, and next with a goat-anti-mouse Ab conjugated to 10 nm gold particles. VLPs adhering to the cell surface were imaged in a Field Emission electron microscope. Samples were scanned for secondary electron (SE) (typical SEM imaging) and backscatter electron mode (BSE). The latter identifies gold particles, which are seen a white dots. As shown in FIG. 7, many filamentous VLPs were found carrying high levels of GCR, as shown by the amount of gold particles attached to the VLPs. The results thus demonstrate the successful formation of VLPs displaying the GCR.

### Example 3—Production Method for RSV-VLPs

For small-scale research, VLPs are typically generated in T flasks, using costly transfection reagents such as lipofectamine2000 (Invitrogen). To improve yield and cost, a protocol was developed that uses polymer-based transfection of HEK 293 Freestyle cells (293-F) in suspension, and medium exchanges to maximize yield. 293-F cells are often used for protein production in suspension, as they grow well in suspension, transfect easily, and yield high protein levels, which can be achieved using serum-free medium.

The developed protocol is described below:

Seed cells: 293-F cells were seeded in sterile erlenmeyers, in commercial serum-free medium (SFM); different sizes of erlenmeyers (or other types of flasks) can be used, with (in certain non-limiting embodiments) a maximum volume of 1 liter suspension. Adaptation to SFM ensures that harvests are free of Fetal Bovine Serum. The cells were grown in a CO₂ shaker at 37° C., 5-10% CO² to a density of about 2 million cells/ml.

Transfect cells: RSV plasmids expressing various combinations of VLP components (N, P, M, and/or F proteins or fragments/variants thereof, etc.) were used. Plasmids were mixed in tubes with the polymer polyethylenimine (PEI), and the cells were spun down and resuspended in transfection medium, then incubated in CO₂ shaker at 37° C. for 3-22 h. Transfection medium was removed by spinning down cells, medium was replaced with SFM, then incubated in CO₂ shaker at 37° C. for another 10-36 hours.

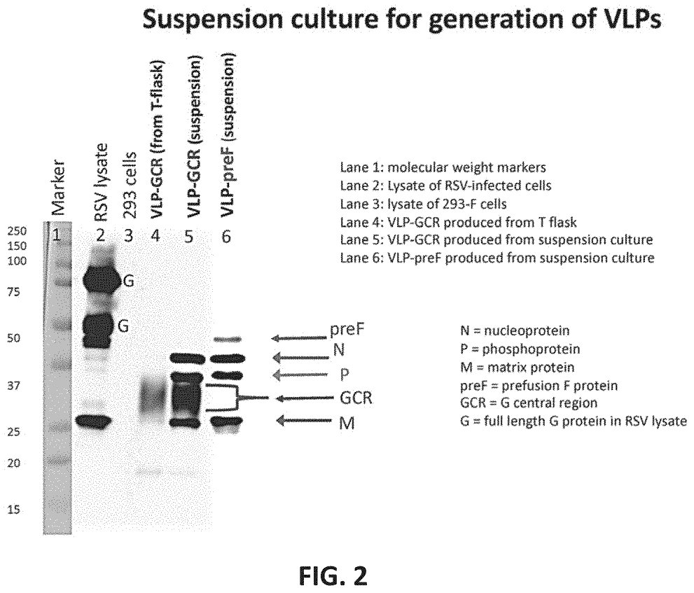

Replace medium to enhance VLP production: at 20-30 hours post start-transfection, cell medium was replaced with a reduced-serum medium (such as OPTIMEM or Advanced DMEM from Life Technologies, Carlsbad, Calif.) without additives but containing an anti-clumping agent (Life Technologies), and further incubated in the CO₂ shaker for 10-20 hours. VLPs were harvested from the supernatant by removing cells in a low-speed centrifugation step, followed by pelleting VLPs through a 25% sucrose cushion. VLP pellets were resuspended in medium without additives. VLP suspension was characterized by western blot (to visualize components; FIG. 2) and Bradford analysis to determine total protein concentration, prior to use as vaccines.

Thus, this non-limiting embodiment of the production protocol is unique in multiple features, including:
adaptation of 293 suspension culture to VLP production by transfecting with multiple plasmids for VLP production;
use of PEI-based transfection for VLP production, reducing cost of production;
use of medium exchange to enhance VLP production—growing transfected cells in media with suboptimal growth conditions appears to enhance VLP production; and
use of shaker culture results in a higher proportion of soluble VLPs, allowing harvest with fewer contaminating proteins (typically RSV particles remain attached to cells and are difficult to purify away from host proteins).

In addition to the above, another option is to modify the 293 production line to enhance VLP production and/or to produce a VLP containing additional RSV or non-RSV components to enhance stability, uptake, or efficacy of the VLPs.

Further, the VLP itself can be modified for various purposes. That is, the VLPs can be modified to improve the stability thereof. Also, the VLPs can be modified to protect the VLP from inactivation by the innate immune response. Further, the VLPs can be modified to target the VLPs to immune cells to modulate the immune response; for example (but not by way of limitation), the VLPs can be modified to target specifically to an immune cell subset, such as, but not limited to, dendritic cells or a dendritic cell subpopulation. Alternatively, the VLPs can be modified to target the VLPs to non-immune cells to modulate the uptake thereof.

### Example 4

The RSV-VLPs of the present disclosure may be utilized in any vaccine regimens known in the art or otherwise contemplated herein. For example, the RSV-VLPs may be delivered in a single dose, or one or more RSV-VLPs (or one or more RSV-VLPs plus other RSV vaccine(s)) may be delivered via multiple doses over a period of time. In this Example, one or two RSV-VLPs of the present disclosure were utilized in a prime-boost type of vaccine regimen, where an immune system of a host is first “primed” with an RSV-VLP and then subsequently delivered a “boost” of the same or a different RSV-VLP.

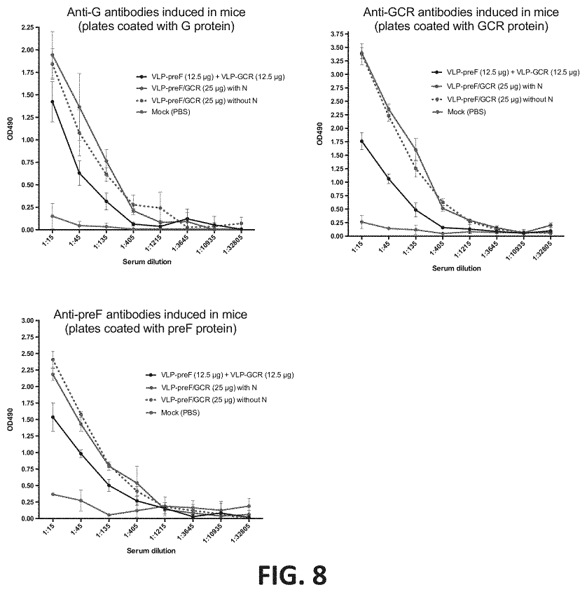

A combination vaccine of preF and GCR VLPs (VLP-preF and VLP-GCR), as well as a VLP containing both preF and GCR in one particle (VLP-preF/GCR) were used to vaccinate mice in a prime-boost regimen. Three weeks post boost, blood samples were taken, and serum antibodies against preF, whole G, and GCR were measured. The results are shown in FIG. 8 and demonstrate that a combination vaccine of VLP-preF and VLP-GCR induced significant levels of anti-preF, anti-G, and anti-GCR antibodies. The results also demonstrated that a VLP containing both preF and GCR elicited higher levels of antibodies following a prime-boost regimen. In addition, these results demonstrated that VLPs with and without N are each capable of inducing high antibody levels.

Therefore, this Example has demonstrated that the VLPs of the present disclosure can be utilized in a prime-boost regimen to induce significant levels of antiviral antibodies.

### Example 5

While Example 4 describes a prime-boost regimen utilizing RSV-VLPs, this Example describes a prime-boost regimen in which the RSV-VLPs of the present disclosure are utilized in combination with a live RSV vaccine candidate. In this prime-boost type of vaccine regimen, an immune system of a host is first “primed” with a live RSV vaccine candidate, and then the RSV-VLPs are subsequently delivered as a “boost” to generate potent and long-term protection against RSV.

One non-limiting example of a live RSV vaccine candidate that can be utilized in accordance with the present disclosure is M-null. M-null is a live vaccine candidate with a stringent safety profile that protects from RSV replication in mice. M-null live, attenuated viruses that can be utilized in accordance with the present disclosure are disclosed in detail in International Patent Application Publication No. WO 2012/167139 and issued U.S. Pat. No. 10,844,357; the entire contents of which are hereby expressly incorporated herein by reference in their entirety. The M-null live, attenuated vaccine induces a promised immune response; however, one major drawback of M-null is that it induces both preF and postF antibodies.

It was previously shown that preF antibodies neutralize better than post F antibodies; however, high levels of (non-neutralizing) postF antibodies can contribute to enhanced lung disease.

The existing M-null vaccine may be utilized in a prime-boost regimen in accordance with the present disclosure. Alternatively, in certain non-limiting aspects, the present Example also considered how M-null could be further improved by focusing the anti-G and anti-F response. For example, the anti-F response can be improved by boosting M-null primed hosts with VLPs carrying only the prefusion form of F (VLP-preF), thereby increasing the proportion of anti-preF antibodies; this in turn will enhance efficacy (i.e., neutralizing potential) and safety (i.e., reduced proportion of postF antibodies). In addition, the anti-G response can be improved by boosting M-null primed hosts with VLPs carrying only the central conserved region of G (G-CCR); this in turn will enhance efficacy and protection across strains.

Focusing the immune response on preF and G-CCR will lower the levels of non-neutralizing antibodies, which were shown to be involved in ERD; focus the G-response on the most relevant region of G (G-CCR), which has a receptor-binding domain, a heparin-binding domain, and which is more conserved between strains (thereby providing protection against different strains)); and focus the F-response on prefusion F (anti-pre-fusion F antibodies were shown to be much more protective in mice than antibodies against the post-fusion form of F). Therefore, the present disclosure also includes a combination of vaccine strategies that include the use of the M-null RSV vaccine (or a variant thereof) with any of the RSV-VLPs disclosed or otherwise contemplated herein.

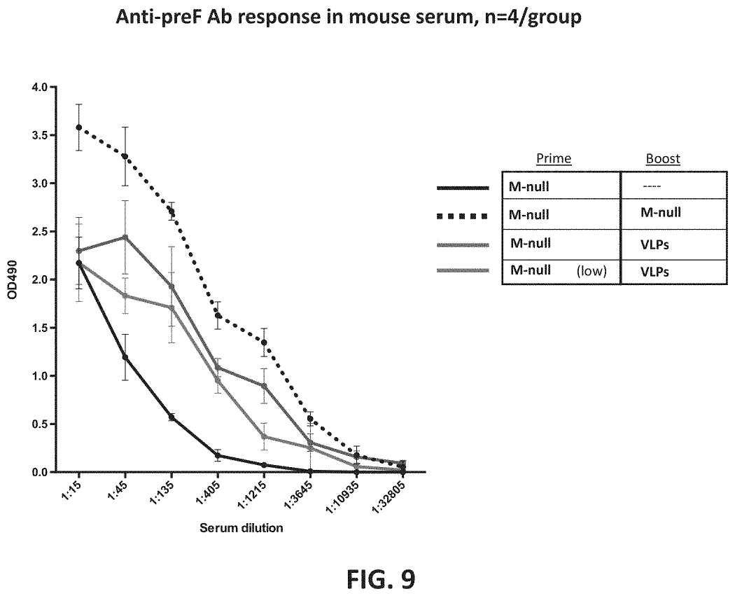

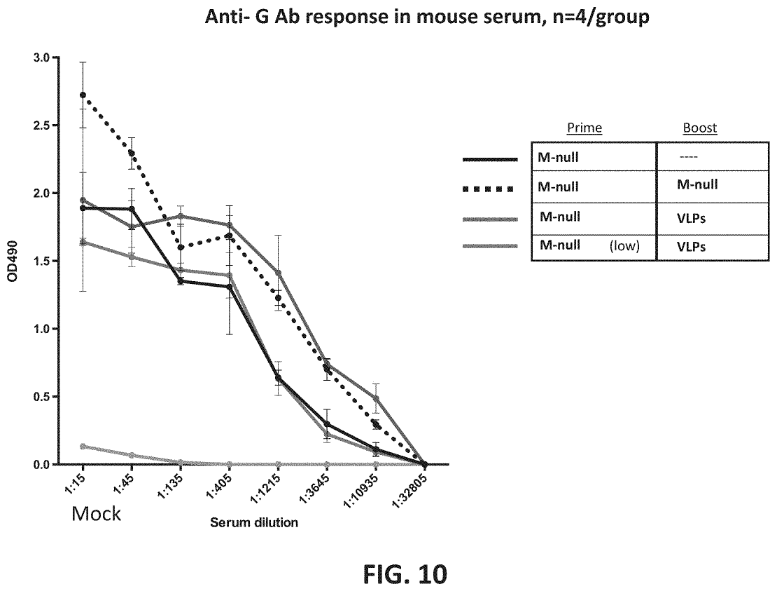

A combined M-null/VLP vaccine strategy was tested in mice. BALB/c female mice were vaccinated with M-null live virus as prime, followed by a boost of VLPs (preF and GCCR). Three weeks post boost, blood samples were taken, and serum antibodies against preF, whole G, and GCR were measured. The results are shown in FIGS. 9, 10, and 11 and demonstrate that a combination prime-boost vaccine regimen of M-null and RSV-VLP induced significant levels of anti-preF, anti-G, and anti-GCR antibodies.

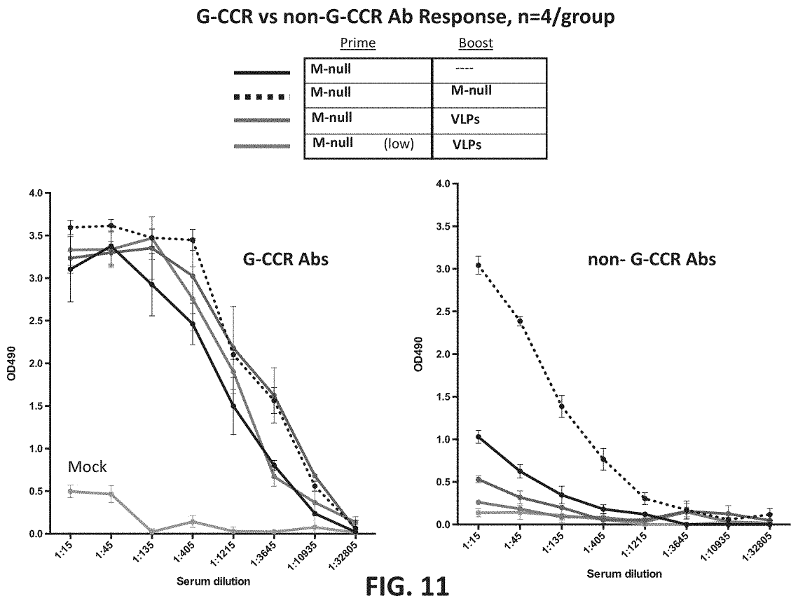

In addition, FIG. 11 demonstrates that a prime-boost regimen that includes VLPs as the boost increases the proportion of G-CCR antibodies produced and decreases the production of non-G-CCR antibodies produced. This antibody response focused on the CCR will confer greater efficacy and safety to the regimen.

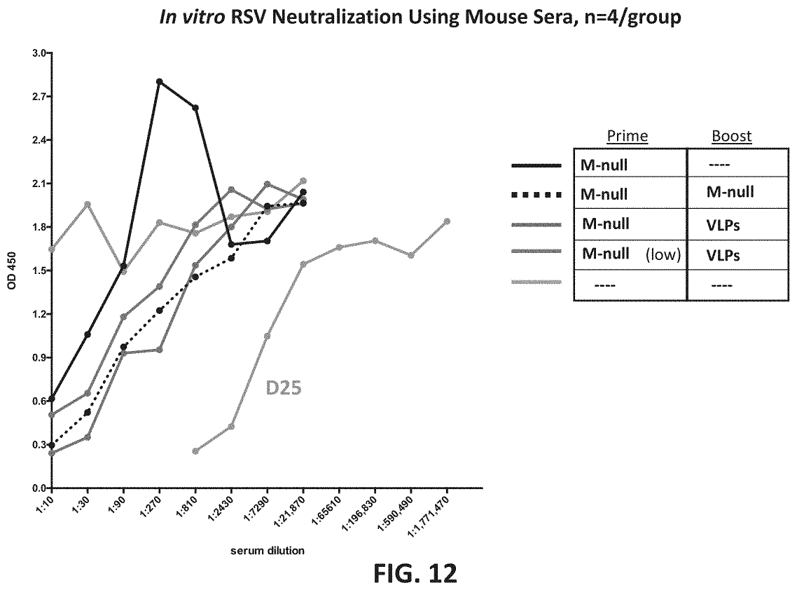

FIG. 12 demonstrates in vitro RSV neutralization analysis using the prime-boost strategies described herein. As can be seen, the prime-boost vaccine regimens of the present disclosure neutralize better than a single dose of M-null vaccine.

The evidence presented herein above demonstrated that boosting with VLP lowers the amount of non-CCRG antibodies and thus increases the proportion of anti-CCRG antibodies as intended. In addition, the evidence demonstrated that boosting with VLPs increased the level of anti-preF antibodies without increasing the level of anti-post-F antibodies, thus also increasing the proportion of anti-preF antibodies. Therefore, the use of RSV-VLPs in combination with M-null focuses the anti-G and anti-F antibody response and thus makes the M-null vaccine more efficacious and safer in humans.

In the infamous failed trial of the 1960s mentioned in the background section, one cause for enhanced disease was too many antibodies that did not neutralize or protect. These non-neutralizing antibodies form immune-complexes and cause damage. As such, there is a need for antibodies that are targeted to the most neutralizing parts of the G and F antigens, which are the GCR and prefusion form of F.

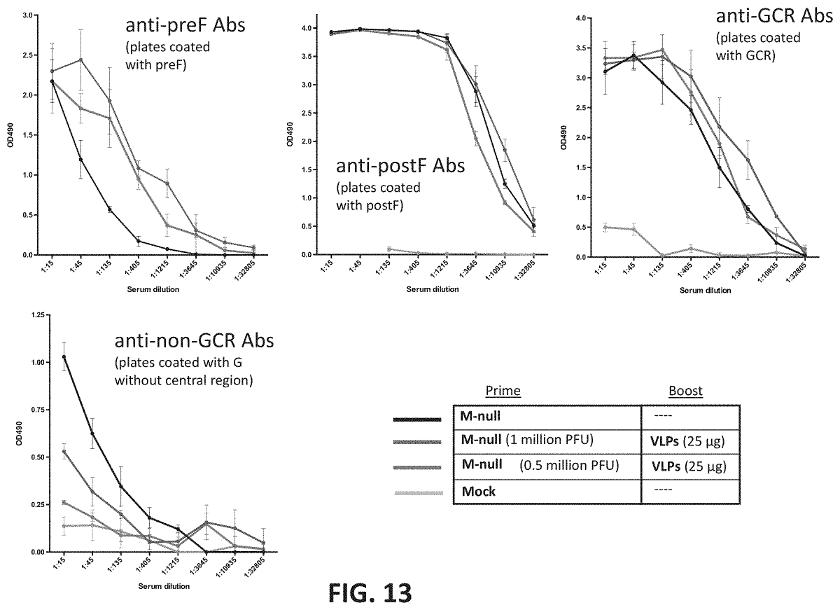

Animals were primed with M-null live vaccine and then boosted with VLP-preF/GCR. Relative to animals that were primed with M-null and not boosted, the prime-boost animal groups showed a strong increase in anti-GCR antibodies and a decrease in anti-non-GCR antibodies (FIG. 13). Thus, boosting with VLPs significantly increased the ratio of preF:postF antibodies and increased the ratio of GCR:non-GCR antibodies. Due to a higher proportion of neutralizing antibodies, the likelihood of immune complexes that enhance lung disease is lower, and thus the safety of the vaccine is enhanced. Therefore, boosting with VLP-preF/GCR after an M-null prime focused the immune response on the most vaccine-relevant regions of RSV (preF and GCR) and improved the efficacy (higher proportion of relevant immunity) and safety (lower immunity against non-protective antigen regions and reduced risk of non-relevant immune complexes).

Thus, in accordance with the present disclosure, there have been provided compositions, as well as methods of producing and using same, which fully satisfy the objectives and advantages set forth hereinabove. Although the present disclosure has been described in conjunction with the specific drawings, experimentation, results, and language set forth hereinabove, it is evident that many alternatives, modifications, and variations will be apparent to those skilled in the art. Accordingly, it is intended to embrace all such alternatives, modifications, and variations that fall within the spirit and broad scope of the present disclosure.

What is claimed is:

1. A respiratory syncytial virus (RSV)-based virus-like particle comprising:
an RSV phosphoprotein (P) or variant or fragment thereof;
an RSV matrix (M) protein or variant or fragment thereof; and
an RSV attachment glycoprotein (G) or variant or fragment thereof, wherein the G protein or variant or fragment thereof comprises at least a portion of a central conserved region of the G protein.

2. The RSV-based virus-like particle of claim 1, wherein the at least a portion of the central conserved region of the G protein comprises a CX₃C domain thereof.

3. The RSV-based virus-like particle of claim 1, wherein the G protein or variant or fragment thereof comprises SEQ ID NO:1.

4. The RSV-based virus-like particle of claim 1, wherein the G protein or variant or fragment thereof further comprises SEQ ID NO:2.

5. The RSV-based virus-like particle of claim 1, wherein the G protein or variant or fragment thereof comprises SEQ ID NO:3.

6. The RSV-based virus-like particle of claim 1, wherein the G protein or variant or fragment thereof is fused to a polypeptide comprising a stem/stalk region of a transmembrane protein.

7. The RSV-based virus-like particle of claim 6, wherein the G protein or variant or fragment thereof is fused to a stem region of an RSV fusion (F) protein variant or fragment thereof.

8. The RSV-based virus-like particle of claim 7, wherein the stem region of the RSV F protein variant or fragment thereof has mutations in amino acids 5573 and N574.

9. The RSV-based virus-like particle of claim 1, wherein the G protein or variant or fragment thereof is a full-length G protein or variant thereof.

10. The RSV-based virus-like particle of claim 1, further comprising an RSV fusion (F) protein variant or fragment thereof, wherein the RSV F protein variant or fragment thereof contains at least one mutation that stabilizes the F protein in pre-fusion form.

11. The RSV-based virus-like particle of claim 10, wherein the RSV F protein variant or fragment thereof is absent at least a portion of a cytoplasmic tail of the native RSV F protein.

12. The RSV-based virus-like particle of claim 1, further comprising an RSV nucleoprotein (N) or variant or fragment thereof.

13. The RSV-based virus-like particle of claim 1, further comprising at least one non-RSV component.

14. An isolated immunogenic composition, comprising:
at least one RSV-based virus-like particle of claim 1.

15. The isolated immunogenic composition of claim 14, further comprising:
at least one RSV-based virus-like particle, comprising:
an RSV phosphoprotein (P) or variant or fragment thereof;
an RSV matrix (M) protein or variant or fragment thereof; and
an RSV fusion (F) protein variant or fragment thereof, wherein the RSV F protein variant or fragment thereof contains at least one mutation that stabilizes the F protein in pre-fusion form.

16. A pharmaceutical composition, comprising:
a therapeutically effective amount of at least one RSV-based virus-like particle of claim 1.

17. The pharmaceutical composition of claim 16, further comprising a pharmaceutically acceptable carrier or excipient.

18. The pharmaceutical composition of claim 16, wherein the pharmaceutical composition is capable of eliciting an immune response against RSV in a mammal.

19. The pharmaceutical composition of claim 16, wherein the therapeutically effective amount of the at least one RSV-based virus-like particle is further defined as an amount sufficient to induce an immune response protective against RSV infection.

20. The pharmaceutical composition of claim 16, wherein the pharmaceutical composition is free of added adjuvants.

21. The pharmaceutical composition of claim 16, further comprising:
a therapeutically effective amount of an RSV-based virus-like particle, comprising:
an RSV phosphoprotein (P) or variant or fragment thereof;
an RSV matrix (M) protein or variant or fragment thereof; and
an RSV fusion (F) protein variant or fragment thereof, wherein the RSV F protein variant or fragment thereof contains at least one mutation that stabilizes the F protein in pre-fusion form.

22. A kit, comprising:
at least one pharmaceutical composition of claim 16.

23. The kit of claim 22, further comprising a second RSV-based virus-like particle, comprising:
an RSV phosphoprotein (P) or variant or fragment thereof;
an RSV matrix (M) protein or variant or fragment thereof; and
an RSV fusion (F) protein variant or fragment thereof, wherein the RSV F protein variant or fragment thereof contains at least one mutation that stabilizes the F protein in pre-fusion form.

24. The kit of claim 22, further comprising a live, attenuated respiratory syncytial virus (RSV).

25. The kit of claim 24, wherein the live, attenuated virus is a recombinant RSV lacking a gene that encodes a matrix (M) protein of the RSV (RSV M-null).

26. The kit of claim 24, wherein the live, attenuated virus is capable of infecting a cell in a mammal but cannot transmit from said cell to another cell in the mammal.

27. A polynucleotide, comprising:
(a) a gene encoding an RSV phosphoprotein (P) or variant or fragment thereof;
(b) a gene encoding an RSV matrix (M) protein or variant or fragment thereof; and
(c) a gene encoding an RSV attachment glycoprotein (G) or variant or fragment thereof, wherein the G protein or variant or fragment thereof comprises at least a portion of a central conserved region of the G protein; and
wherein at least one of (a)-(c) has been codon-optimized.

28. The polynucleotide of claim 27, wherein the at least a portion of the central conserved region of the G protein comprises a CX₃C domain thereof.

29. The polynucleotide of claim 27, wherein the G protein or variant or fragment thereof comprises SEQ ID NO:1.

30. The polynucleotide of claim 27, wherein the G protein or variant or fragment thereof of (c) further comprises SEQ ID NO:2.

31. The polynucleotide of claim 27, further defined as comprising at least one of SEQ ID NOs:5, 7, 9, 11, 13, 15, 17, 19, and 21.

32. A vector encoding at least a portion of at least one RSV-based virus-like particle, the polynucleotide comprising:
(a) a polynucleotide encoding an RSV phosphoprotein (P) or variant or fragment thereof;
(b) a polynucleotide encoding an RSV matrix (M) protein or variant or fragment thereof; and
(c) a polynucleotide encoding an RSV attachment glycoprotein (G) or variant or fragment thereof, wherein the G protein or variant or fragment thereof comprises at least a portion of a central conserved region of the G protein.

33. The vector of claim 32, wherein at least one of polynucleotides (a)-(c) has been codon-optimized.

34. The vector of claim 32, wherein the at least a portion of the central conserved region of the G protein comprises a CX₃C domain thereof.

35. The vector of claim 32, wherein the G protein or variant or fragment thereof comprises SEQ ID NO:1.

36. The vector of claim 32, wherein the G protein or variant or fragment thereof of (c) further comprises SEQ ID NO:2.

37. The vector of claim 32, further defined as comprising at least one of SEQ ID NOs:5, 7, 9, 11, 13, 15, 17, 19, and 21.

38. A mammalian cell, comprising:
at least one vector of claim 32; and
wherein the cell produces at least one RSV-based virus-like particle.

39. The mammalian cell of claim 38, further defined as a 293 cell.

40. A method of producing at least one RSV-based virus-like particle, the method comprising the steps of:
culturing a cell line that expresses at least one RSV-based virus-like particle of claim 1, wherein the cell line is cultured under conditions that allow for production of the at least one RSV-based virus-like particle; and
recovering the at least one RSV-based virus-like particle.

41. A method, comprising the step of:
administering at least one pharmaceutical composition of claim 16 to the mammal.

42. The method of claim 41, wherein the at least one pharmaceutical composition is administered or introduced intranasally.

43. The method of claim 41, further comprising the step of:
administering to the mammal a live, attenuated respiratory syncytial virus.

44. The method of claim 43, wherein the live, attenuated virus is administered to the mammal prior to the at least one pharmaceutical composition.

45. The method of claim 43, wherein the live, attenuated RSV is a recombinant RSV lacking a gene that encodes a matrix (M) protein of the RSV (RSV M-null).

46. The method of claim 43, wherein the virus is capable of infecting a cell in a mammal but cannot transmit from said cell to another cell in the mammal.

47. The method of claim 43, wherein no adjuvants are administered to the mammal in the method.

48. The method of claim 41, further comprising the step of administering at least one additional pharmaceutical composition to the mammal, wherein the at least one additional pharmaceutical composition comprises a second RSV-based virus-like particle, comprising:
an RSV phosphoprotein (P) or variant or fragment thereof;
an RSV matrix (M) protein or variant or fragment thereof; and
an RSV fusion (F) protein variant or fragment thereof, wherein the RSV F protein variant or fragment thereof contains at least one mutation that stabilizes the F protein in pre-fusion form.

49. The method of claim 48, wherein the at least two pharmaceutical compositions are administered simultaneously.

50. The method of claim 48, wherein the at least two pharmaceutical compositions are administered wholly or partially sequentially.

51. The method of claim 41, further defined as a method of eliciting an immune response in a mammal.

52. The method of claim 41, further defined as a method of generating antibodies specific for RSV in a mammal.

53. The method of claim 41, further defined as a method of reducing the occurrence or severity of respiratory syncytial virus infection in a mammal.

54. The method of claim 53, wherein the mammal has previously been immunized with a live, attenuated respiratory syncytial virus.


SEQUENCE LISTING (22 sequences)

SEQ ID NO: 1 - protein, 14 aa, respiratory syncytial virus
CSICSNNPTCWAIC

SEQ ID NO: 2 - protein, 14 aa, respiratory syncytial virus
ICKRIPNKKPGKKT

SEQ ID NO: 3 - protein, 75 aa, respiratory syncytial virus
TTTQTQPSKPTTKQRQNKPPSKPNNDFHFEVFNFVPCSICSNNPTCWAICKRIPNKKPGKKTTTKPTKKPTLKTT

SEQ ID NO: 4 - DNA, 1176 bp, Artificial sequence
  Codon-optimized sequence of RSV N sequence
atggccctgagcaaagtgaagctgaacgacaccctgaacaaggaccagctgctgtcctccagcaagtacaccatccagcgcagcaccggcgacagcatcgacacccccaactacgacgtgcagaagcacatcaacaagctgtgcggcatgctgctgatcaccgaggacgccaaccacaagttcaccggcctgatcggcatgctgtacgccatgtcccgcctgggccgcgaggacaccatcaagatcctgcgcgacgccggctaccacgtgaaggccaacggcgtggacgtgaccacccaccgccaagacatcaacggcaaggagatgaagttcgaagtgctgaccctggccagcctgaccaccgagatccagatcaacatcgagatcgagagccgcaagagctacaagaagatgctgaaggagatgggcgaagtggcccccgagtaccgccacgacagccccgactgcggcatgatcatcctgtgcatcgccgccctggtgatcaccaagctggccgccggcgaccgcagcggcctgaccgccgtgatccgccgcgccaacaacgtgctgaagaacgagatgaagcgctacaagggcctgctgcccaaggacatcgccaacagcttctacgaagtgttcgagaagcacccccacttcatcgacgtgttcgtgcacttcggcatcgcccagagcagcacccgcggcggcagccgcgtggagggcatcttcgccggcctgttcatgaacgcctacggcgccggccaagtgatgctgcgctggggcgtgctggccaagagcgtgaagaacatcatgctgggccacgccagcgtgcaagccgagatggagcaagtggtggaagtgtacgagtacgcccagaagctgggcggcgaggccggcttctaccacatcctgaacaaccccaaggccagcctgctgagcctgacccagttcccccacttcagcagcgtggtgctgggaaacgccgccggcctgggcatcatgggcgagtaccgcggcaccccccgcaaccaagacctgtacgacgccgccaaggcctacgccgagcagctgaaggagaacggcgtgatcaactacagcgtgctggacctgaccgccgaggagctggaggccatcaagcaccagctgaaccccaaggacaacgacgtggagctgtaa

SEQ ID NO: 5 - protein, 391 aa, respiratory syncytial virus
MALSKVKLNDTLNKDQLLSSSKYTIQRSTGDSIDTPNYDVQKHINKLCGMLLITEDANHKFTGLIGMLYAMSRLGREDTIKILRDAGYHVKANGVDVTTHRQDINGKEMKFEVLTLASLTTEIQINIEIESRKSYKKMLKEMGEVAPEYRHDSPDCGMIILCIAALVITKLAAGDRSGLTAVIRRANNVLKNEMKRYKGLLPKDIANSFYEVFEKHPHFIDVFVHFGIAQSSTRGGSRVEGIFAGLFMNAYGAGQVMLRWGVLAKSVKNIMLGHASVQAEMEQVVEVYEYAQKLGGEAGFYHILNNPKASLLSLTQFPHFSSVVLGNAAGLGIMGEYRGTPRNQDLYDAAKAYAEQLKENGVINYSVLDLTAEELEAIKHQLNPKDNDVEL

SEQ ID NO: 6 - DNA, 1200 bp, Artificial sequence
  RSV N/flag sequence - codon-optimized RSV N sequence additionally encoding a flag tag at amino acid position 2
atggactacaaggacgacgacgacaaggccctgagcaaagtgaagctgaacgacaccctgaacaaggaccagctgctgtcctccagcaagtacaccatccagcgcagcaccggcgacagcatcgacacccccaactacgacgtgcagaagcacatcaacaagctgtgcggcatgctgctgatcaccgaggacgccaaccacaagttcaccggcctgatcggcatgctgtacgccatgtcccgcctgggccgcgaggacaccatcaagatcctgcgcgacgccggctaccacgtgaaggccaacggcgtggacgtgaccacccaccgccaagacatcaacggcaaggagatgaagttcgaagtgctgaccctggccagcctgaccaccgagatccagatcaacatcgagatcgagagccgcaagagctacaagaagatgctgaaggagatgggcgaagtggcccccgagtaccgccacgacagccccgactgcggcatgatcatcctgtgcatcgccgccctggtgatcaccaagctggccgccggcgaccgcagcggcctgaccgccgtgatccgccgcgccaacaacgtgctgaagaacgagatgaagcgctacaagggcctgctgcccaaggacatcgccaacagcttctacgaagtgttcgagaagcacccccacttcatcgacgtgttcgtgcacttcggcatcgcccagagcagcacccgcggcggcagccgcgtggagggcatcttcgccggcctgttcatgaacgcctacggcgccggccaagtgatgctgcgctggggcgtgctggccaagagcgtgaagaacatcatgctgggccacgccagcgtgcaagccgagatggagcaagtggtggaagtgtacgagtacgcccagaagctgggcggcgaggccggcttctaccacatcctgaacaaccccaaggccagcctgctgagcctgacccagttcccccacttcagcagcgtggtgctgggaaacgccgccggcctgggcatcatgggcgagtaccgcggcaccccccgcaaccaagacctgtacgacgccgccaaggcctacgccgagcagctgaaggagaacggcgtgatcaactacagcgtgctggacctgaccgccgaggagctggaggccatcaagcaccagctgaaccccaaggacaacgacgtggagctgtaa

SEQ ID NO: 7 - protein, 399 aa, Artificial sequence
  Amino acid sequence of SEQ ID NO6 (RSV N/flag)
MDYKDDDDKALSKVKLNDTLNKDQLLSSSKYTIQRSTGDSIDTPNYDVQKHINKLCGMLLITEDANHKFTGLIGMLYAMSRLGREDTIKILRDAGYHVKANGVDVTTHRQDINGKEMKFEVLTLASLTTEIQINIEIESRKSYKKMLKEMGEVAPEYRHDSPDCGMIILCIAALVITKLAAGDRSGLTAVIRRANNVLKNEMKRYKGLLPKDIANSFYEVFEKHPHFIDVFVHFGIAQSSTRGGSRVEGIFAGLFMNAYGAGQVMLRWGVLAKSVKNIMLGHASVQAEMEQVVEVYEYAQKLGGEAGFYHILNNPKASLLSLTQFPHFSSVVLGNAAGLGIMGEYRGTPRNQDLYDAAKAYAEQLKENGVINYSVLDLTAEELEAIKHQLNPKDNDVEL

SEQ ID NO: 8 - DNA, 726 bp, Artificial sequence
  Codon-optimized sequence of RSV P sequence
atggagaagttcgcccccgagttccacggcgaggacgccaacaaccgcgccaccaagttcctggagagcatcaagggtaagttcaccagccccaaggaccccaagaagaaggacagcatcatcagcgtgaacagcatcgacatcgaagtgaccaaggagagccccatcaccagcaacagcaccatcatcaaccccaccaacgagaccgacgacaccgccggcaacaagcccaactaccagcgcaagcccctggtgagcttcaaggaggaccccacccccagcgacaaccccttcagcaagctctacaaggagaccatcgagaccttcgacaacaacgaggaggagagcagctacagctacgaggagatcaacgaccagaccaacgacaacatcaccgcccgcctggaccgcatcgacgagaagctgagcgagatcctgggcatgctgcacaccctggtggtggccagcgccggccccaccagcgcccgcgacggcatccgcgacgctatggtgggcctgcgcgaggagatgatcgagaagatccgcaccgaggccctgatgaccaacgaccgcctggaggctatggcccgcctgcgcaacgaggagagcgagaagatggccaaggacaccagcgacgaagtgagcctgaaccccaccagcgagaagctgaacaacctgctggagggtaacgacagcgacaacgacctgagcctggaggacttctaa

SEQ ID NO: 9 - protein, 241 aa, respiratory syncytial virus
MEKFAPEFHGEDANNRATKFLESIKGKFTSPKDPKKKDSIISVNSIDIEVTKESPITSNSTIINPTNETDDTAGNKPNYQRKPLVSFKEDPTPSDNPFSKLYKETIETFDNNEEESSYSYEEINDQTNDNITARLDRIDEKLSEILGMLHTLVVASAGPTSARDGIRDAMVGLREEMIEKIRTEALMTNDRLEAMARLRNEESEKMAKDTSDEVSLNPTSEKLNNLLEGNDSDNDLSLEDF

SEQ ID NO: 10 - DNA, 726 bp, Artificial sequence
  RSV P-mut sequence codon-optimized, and has amino acids 43 and 54 mutated to alanine
atggagaagttcgcccccgagttccacggcgaggacgccaacaaccgcgccaccaagttcctggagagcatcaagggtaagttcaccagccccaaggaccccaagaagaaggacagcatcatcagcgccaacagcatcgacatcgaagtgaccaaggaggcccccatcaccagcaacagcaccatcatcaaccccaccaacgagaccgacgacaccgccggcaacaagcccaactaccagcgcaagcccctggtgagcttcaaggaggaccccacccccagcgacaaccccttcagcaagctctacaaggagaccatcgagaccttcgacaacaacgaggaggagagcagctacagctacgaggagatcaacgaccagaccaacgacaacatcaccgcccgcctggaccgcatcgacgagaagctgagcgagatcctgggcatgctgcacaccctggtggtggccagcgccggccccaccagcgcccgcgacggcatccgcgacgctatggtgggcctgcgcgaggagatgatcgagaagatccgcaccgaggccctgatgaccaacgaccgcctggaggctatggcccgcctgcgcaacgaggagagcgagaagatggccaaggacaccagcgacgaagtgagcctgaaccccaccagcgagaagctgaacaacctgctggagggtaacgacagcgacaacgacctgagcctggaggacttctaa

SEQ ID NO: 11 - protein, 241 aa, Artificial sequence
  Amino acid sequence of SEQ ID NO10
MEKFAPEFHGEDANNRATKFLESIKGKFTSPKDPKKKDSIISANSIDIEVTKEAPITSNSTIINPTNETDDTAGNKPNYQRKPLVSFKEDPTPSDNPFSKLYKETIETFDNNEEESSYSYEEINDQTNDNITARLDRIDEKLSEILGMLHTLVVASAGPTSARDGIRDAMVGLREEMIEKIRTEALMTNDRLEAMARLRNEESEKMAKDTSDEVSLNPTSEKLNNLLEGNDSDNDLSLEDF

SEQ ID NO: 12 - DNA, 747 bp, Artificial sequence
  RSV P/flag-mut sequence RSV P sequence odon-optimized, amino acids 43 and 54 mutated to alanine, and a flag tag at position 241
atggagaagttcgcccccgaattccacggcgaggacgccaacaaccgcgccaccaagttcctggagagcatcaagggtaagttcaccagccccaaggaccccaagaagaaggacagcatcatcagcgccaacagcatcgacatcgaagtgaccaaggaggcccccatcaccagcaacagcaccatcatcaaccccaccaacgagaccgacgacaccgccggcaacaagcccaactaccagcgcaagcccctggtgagcttcaaggaggaccccacccccagcgacaaccccttcagcaagctctacaaggagaccatcgagaccttcgacaacaacgaggaggagagcagctacagctacgaggagatcaacgaccagaccaacgacaacatcaccgcccgcctggaccgcatcgacgagaagctgagcgagatcctgggcatgctgcacaccctggtggtggccagcgccggccccaccagcgcccgcgacggcatccgcgacgctatggtgggcctgcgcgaggagatgatcgagaagatccgcaccgaggccctgatgaccaacgaccgcctggaggctatggcccgcctgcgcaacgaggagagcgagaagatggccaaggacaccagcgacgaagtgagcctgaaccccaccagcgagaagctgaacaacctgctggagggtaacgacagcgacaacgacctgagcctggaggactacaaggacgacgacgacaagttctaa

SEQ ID NO: 13 - protein, 248 aa, Artificial sequence
  Amino acid sequence of SEQ ID NO12
MEKFAPEFHGEDANNRATKFLESIKGKFTSPKDPKKKDSIISANSIDIEVTKEAPITSNSTIINPTNETDDTAGNKPNYQRKPLVSFKEDPTPSDNPFSKLYKETIETFDNNEEESSYSYEEINDQTNDNITARLDRIDEKLSEILGMLHTLVVASAGPTSARDGIRDAMVGLREEMIEKIRTEALMTNDRLEAMARLRNEESEKMAKDTSDEVSLNPTSEKLNNLLEGNDSDNDLSLEDYKDDDDKF

SEQ ID NO: 14 - DNA, 771 bp, Artificial sequence
  RSV M sequence codon-optimized
atggagacctacgtgaacaagctgcacgagggcagcacctacaccgccgccgtgcagtacaacgtgctggagaaggacgacgaccccgccagcctgaccatctgggtgcccatgttccagagcagcatgcccgccgacctgctgatcaaggagctggccaacgtgaacatcctggtgaagcagatcagcacccccaaggggcctagcctgcgcgtgatgatcaacagccgcagcgccgtgctggcccagatgcccagcaagttcaccatctgcgccaacgtgagcctggacgagcgcagcaagctggcctacgacgtgaccaccccctgcgagatcaaggcctgcagcctgacctgcctgaagagcaagaacatgctgaccaccgtgaaggacctgaccatgaagaccctgaaccccacccacgacatcatcgccctgtgcgagttcgagaacatcgtgaccagcaagaaagtgatcatccccacctacctgcgcagcatcagcgtgcgcaacaaggacctgaacaccctggagaacatcaccaccaccgagttcaagaacgccatcaccaacgccaagatcatcccctacagcggcctgctgctggtgatcaccgtgaccgacaacaagggcgccttcaagtacatcaagccccagagccagttcatcgtggacctgggcgcctacctggagaaggagagcatctactacgtgaccaccaactggaagcacaccgccacccgcttcgccatcaagcctatggaggactaa

SEQ ID NO: 15 - protein, 256 aa, respiratory syncytial virus
METYVNKLHEGSTYTAAVQYNVLEKDDDPASLTIWVPMFQSSMPADLLIKELANVNILVKQISTPKGPSLRVMINSRSAVLAQMPSKFTICANVSLDERSKLAYDVTTPCEIKACSLTCLKSKNMLTTVKDLTMKTLNPTHDIIALCEFENIVTSKKVIIPTYLRSISVRNKDLNTLENITTTEFKNAITNAKIIPYSGLLLVITVTDNKGAFKYIKPQSQFIVDLGAYLEKESIYYVTTNWKHTATRFAIKPMED

SEQ ID NO: 16 - DNA, 1725 bp, Artificial sequence
  RSV preF sequence codon-optimized, and final two amino acids mutated to alanine
atggagctgctgatcctgaaggccaacgccatcaccaccatcctgaccgccgtgaccttctgcttcgccagcggccagaacatcaccgaggagttctaccagagcacctgcagcgccgtgagcaagggctacctgagcgccctgcgcaccggctggtacaccagcgtgatcaccatcgagctgagcaacatcaaggagaacaagtgcaacggcaccgacgccaaagtgaagctgatcaagcaagagctggacaagtacaagaacgccgtgaccgagctgcagctgctgacccagagcacccccgccaccaacaaccgggcccgccgcgagctgccccgcttcatgaactacaccctgaacaacgccaagaagaccaacgtgaccctgagcaagaagcgcaagcgccgcttcctgggcttcctgctgggcgtgggcagcgccatcgccagcggcgtggccgtgtgtaaagtgctgcacctggagggcgaagtgaacaagatcaagagcgccctgctgagcaccaacaaggccgtggtgagcctgagcaacggcgtgagcgtgctgaccttcaaagtgctggacctgaagaactacatcgacaagcagctgctgcccatcctcaacaagcagagctgcagcatcagcaacatcgagaccgtgatcgagttccagcagaagaacaaccgcctgctggagatcacccgcgagttcagcgtgaacgccggcgtgaccacccccgtgagcacctacatgctgaccaacagcgagctgctgagcctgatcaacgacatgcccatcaccaacgaccagaagaagctgatgagcaacaacgtgcagatcgtgcgccagcagagctacagcatcatgtgtatcatcaaggaggaagtgctggcctacgtggtgcagctgcccctgtacggcgtgatcgacaccccctgctggaagctgcacaccagccccctgtgcaccaccaacaccaaggagggcagcaacatctgcctgacgcgtaccgaccgcggctggtactgcgacaacgccggcagcgtgagcttcttcccccaagccgagacctgcaaagtgcagagcaaccgcgtgttctgcgacaccatgaacagcctgaccctgcccagcgaagtgaacctgtgcaacgtggacatcttcaaccccaagtacgactgcaagatcatgaccagcaagaccgacgtgagcagcagcgtgatcaccagcctgggcgccatcgtgagctgctacgggaagaccaagtgcaccgccagcaacaagaaccgcggcatcatcaagaccttcagcaacggctgcgactacgtgagcaacaagggcgtggacaccgtgagcgtggggaacaccctgtactacgtgaacaagcaagaggggaagagcctgtacgtgaagggcgagcccatcatcaacttctacgaccccctggtgttccccagcgacgagttcgacgccagcatcagccaagtgaacgagaagatcaaccagagtctggccttcatccgcaagagcgacgagctgctgcacaacgtgaacgccgggaagagcaccaccaacatcatgatcaccaccatcatcatcgtgatcatcgtgatcctgctgagcctgatcgccgtgggcctgctgctgtactgcaaggcccgcagcacccccgtgaccctgagcaaggaccagctgagcggcatcaacaacatcgccttcgccgcctaa

SEQ ID NO: 17 - protein, 574 aa, Artificial sequence
  Amino acid sequence of SEQ ID NO16
MELLILKANAITTILTAVTFCFASGQNITEEFYQSTCSAVSKGYLSALRTGWYTSVITIELSNIKENKCNGTDAKVKLIKQELDKYKNAVTELQLLTQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFAA

SEQ ID NO: 18 - DNA, 570 bp, Artificial sequence
  Fstem-GCR sequence RSV sequence codon- optimized and mutated.
atggagctgctgatcctgaaagtgaacgccatcaccaccatcctgaccgccgtgaccttctgcttcgccagcggccaggccatcaccgaggaattctaccagagcaccaccacccagacccagcccagcaagcccaccaccaagcagcgccagaacaagcctcccagcaagcccaacaacgacttccacttcgaagtgttcaacttcgtgccttgcagcatttgcagcaacaaccccacttgttgggccatttgcaagcgcatccccaacaagaagcccggaaagaagaccaccaccaagcccaccaagaagcccaccctcaagaccaccaacgagaagatcacccagagtctggccttcatccgcaagagcgacgagctgctgcacaacgtgaacgccgggaagagcaccaccaacatcatgatcaccaccatcatcatcgtgatcatcgtgatcctgctgagcctgatcgccgtgggcctgctgctgtactgcaaggcccgcagcacccccgtgaccctgagcaaggaccagctgagcggcatcaacaacatcgccttcgccgcctaa

SEQ ID NO: 19 - protein, 189 aa, Artificial sequence
  Amino acid sequence of SEQ ID NO18
MELLILKVNAITTILTAVTFCFASGQAITEEFYQSTTTQTQPSKPTTKQRQNKPPSKPNNDFHFEVFNFVPCSICSNNPTCWAICKRIPNKKPGKKTTTKPTKKPTLKTTNEKITQSLAFIRKSDELLHNVNAGKSTTNIMITTIIIVIIVILLSLIAVGLLLYCKARSTPVTLSKDQLSGINNIAFAA

SEQ ID NO: 20 - DNA, 462 bp, Artificial sequence
  RSV NGFR-GCR sequence codon-optimized and mutated
atgggggcaggtgccaccggccgcgccatggacgggccgcgcctgctgctgttgctgcttctgggggtgtcccttggaggtgccaaggaggcaaccaccacccagacccagcccagcaagcccaccaccaagcagcgccagaacaagcctcccagcaagcccaacaacgacttccacttcgaagtgttcaacttcgtgccttgcagcatttgcagcaacaaccccacttgttgggccatttgcaagcgcatccccaacaagaagcccggaaagaagaccaccaccaagcccaccaagaagcccaccctcaagaccaccgtggcaggtgtggtgaccacagtgatgggcagctcccagcccgtggtgacccgaggcaccaccgacaacctcatccctgtctattgctccatcctggctgctgtggttgtgggtcttgtggcctacatagccttcaagaggtga

SEQ ID NO: 21 - protein, 153 aa, Artificial sequence
  Amino acid sequence of SEQ ID NO20
MGAGATGRAMDGPRLLLLLLLGVSLGGAKEATTTQTQPSKPTTKQRQNKPPSKPNNDFHFEVFNFVPCSICSNNPTCWAICKRIPNKKPGKKTTTKPTKKPTLKTTVAGVVTTVMGSSQPVVTRGTTDNLIPVYCSILAAVVVGLVAYIAFKR

SEQ ID NO: 22 - protein, 5 aa, respiratory syncytial virus
CWAIC